(19) United States
(12) Patent Application Publication
(10) Pub. No.: US 2025/0353923 A1
(43) Pub. Date: Nov. 20, 2025

(54) ANTI-CD38 FUSION PROTEIN FORMULATION

(71) Applicants: Takeda Pharmaceutical Company Limited, Osaka-shi, Osaka (JP); Cephalon LLC, West Chester, PA (US)

(72) Inventors: Tomomi Sato, Fujisawa-Shi, Kanagawa (JP); Nobel Truong, Cambridge, MA (US); Keethkumar Jain, Lexington, MA (US); Nazila Salamat-Miller, Lexington, MA (US); Goutham Kodali, West Chester, PA (US); Shyam Mehta, West Chester, PA (US)

(73) Assignees: Takeda Pharmaceutical Company Limited, Osaka-shi, Osaka (JP); Cephalon LLC, West Chester, PA (US)

(21) Appl. No.: 18/866,286

(22) PCT Filed: May 17, 2023
(86) PCT No.: PCT/IB2023/000287

§ 371 Date: Nov. 15, 2024

Related U.S. Application Data
(60) Provisional application No. 63/343,486, filed on May 18, 2022.

(51) Int. Cl. C07K 16/28; A61K 9/19; A61K 38/00; A61K 45/06; A61K 47/18; A61K 47/22; A61K 47/26; C07K 14/56

(52) U.S. Cl. CPC C07K 16/2896; A61K 9/19; A61K 45/06; A61K 47/183; A61K 47/22; A61K 47/26; C07K 14/56; A61K 38/00; C07K 2317/52; C07K 2317/565; C07K 2319/00

(57) ABSTRACT

Disclosed herein are compositions comprising a CD38-binding fusion protein, a buffer (e.g., a histidine/histidine-HCl buffer), a tonicity agent (e.g., arginine-HCl), a stabilizer (e.g., sucrose), and a surfactant (e.g., polysorbate such as polysorbate 80). In some aspects, the compositions described herein provide for stable storage of the CD38-binding fusion protein when lyophilized. Methods of treating cancer (e.g., CD38 positive cancer) using the composition described herein are also provided.

### RELATED APPLICATIONS

This application claims the benefit under 35 U.S.C. § 119(e) of U.S. provisional application No. 63/343,486, titled “ANTI-CD38 FUSION PROTEIN FORMULATION,” filed May 18, 2022, which is incorporated by reference herein in its entirety.

### BACKGROUND

CD38 is a 46 kDa type II transmembrane glycoprotein. It has a short N-terminal cytoplasmic tail of 20 amino acids, a single transmembrane helix and a long extracellular domain of 256 amino acids. It is expressed on the surface of many immune cells including CD4 and CD8 positive T cells, B cells, NK cells, monocytes, plasma cells, and on a significant proportion of normal bone marrow precursor cells. CD38 is expressed at high levels on various types of cancer cells, e.g., multiple myeloma cells, in most cases of T-and B-lineage acute lymphoblastic leukemias, some acute myelocytic leukemias, follicular center cell lymphomas and T lymphoblastic lymphomas. CD38 is also expressed on B-lineage chronic lymphoblastic leukemia (B-CLL) cells. Antibodies that target CD38 have been used in the treatment of CD38-expressing cancers and hematological malignancies.

Interferons, and in particular IFN-alpha, are able to increase apoptosis and decrease proliferation of certain cancer cells. IFN-alpha has been approved by the FDA for the treatment of several cancers including melanoma, renal cell carcinoma, B cell lymphoma, multiple myeloma, chronic myelogenous leukemia (CML) and hairy cell leukemia. In general, IFN may be targeted to cancer cells, for example, by linking it with a targeting antibody or targeting fragment thereof.

Fusion proteins containing anti-CD38 antibodies fused to IFN-alpha and their use in treating cancer have been described.

### SUMMARY

The present disclosure, in some aspects, relates to compositions comprising a CD38-binding fusion protein, wherein the CD38-binding fusion comprises an anti-CD38 antibody fused to an attenuated interferon alpha-2b protein. In some embodiments, a composition comprising a CD38-binding fusion protein described herein further comprises a buffer, a tonicity agent, a stabilizer, and a surfactant. In some embodiments, a CD38-binding fusion protein described herein is stable and/or remains active in the composition (e.g., when stored for periods of months to years). In some embodiments, a composition described herein is an aqueous solution. In some embodiments, a composition described herein is in lyophilized form. Methods of using the compositions described herein for treating cancer are also provided.

In some aspects this application discloses a composition comprising a CD38-binding fusion protein, a buffer, a tonicity agent, a stabilizer, and a surfactant, wherein the CD38-binding fusion protein comprises an anti-CD38 antibody fused to an attenuated interferon alpha-2b.

In some embodiments, the anti-CD38 antibody comprises a heavy chain complementarity determining region 1 (CDR-H1) comprising the amino acid sequence of SEQ ID NO: 1, a heavy chain complementarity determining region 2 (CDR-H2) comprising the amino acid sequence of SEQ ID NO: 2, a heavy chain complementarity determining region 3 (CDR-H3) comprising the amino acid sequence of SEQ ID NO: 3, a light chain complementarity determining region 1 (CDR-L1) comprising the amino acid sequence of SEQ ID NO: 4, a light chain complementarity determining region 2 (CDR-L2) comprising the amino acid sequence of SEQ ID NO: 5, a light chain complementarity determining region 3 (CDR-L3) comprising the amino acid sequence of SEQ ID NO: 6.

In some embodiments, the anti-CD38 antibody comprises a heavy chain variable region comprising the amino acid sequence of SEQ ID NO: 7 and a light chain variable region comprising the amino acid sequence of SEQ ID NO: 8.

In some embodiments, the anti-CD38 antibody comprises a human IgG4 constant region. In some embodiments, the human IgG4 constant region comprises a proline at position 228 according to the EU numbering system. In some embodiments, the human IgG4 constant region further comprises a tyrosine at position 252, a threonine at position 254, and a glutamic acid at position 256 of the constant region according to the EU numbering system. In some embodiments, the anti-CD38 antibody comprises a heavy chain comprising the amino acid sequence of SEQ ID NO: 9 and a light chain comprising the amino acid sequence of SEQ ID NO: 10.

In some embodiments, the attenuated interferon alpha-2b comprises T106A and A145D mutations relative to an interferon alpha-2b comprising the amino acid sequence of SEQ ID NO: 11.

In some embodiments, the attenuated interferon alpha-2b comprises the amino acid sequence of SEQ ID NO: 12. In some embodiments, the attenuated interferon alpha-2b is fused to the C-terminus of the heavy chain. In some embodiments, the CD38-binding fusion protein comprises a heavy chain comprising the amino acid sequence of SEQ ID NO: 13 and a light chain comprising the amino acid sequence of SEQ ID NO: 10.

In some embodiments, the composition comprises the CD38-binding fusion protein at a concentration of about 8.5-100 mg/ml. In some embodiments, the composition comprises the CD38-binding fusion protein at a concentration of about 30-100 mg/ml. In some embodiments, the composition comprises the CD38-binding fusion protein at a concentration of about 30-70 mg/ml. In some embodiments, the composition comprises the CD38-binding fusion protein at a concentration of about 30 mg/ml. In some embodiments, the composition comprises the CD38-binding fusion protein at a concentration of about 40 mg/ml. In some embodiments, the composition comprises the CD38-binding fusion protein at a concentration of about 60 mg/ml. In some embodiments, the composition comprises the CD38-binding fusion protein at a concentration of about 80 mg/ml.

In some embodiments, the composition comprises the CD38-binding fusion protein at a concentration of about 8.5-11.5 mg/ml. In some embodiments, the composition comprises the CD38-binding fusion protein at a concentration of about 10 mg/ml. In some embodiments, the buffer comprises histidine and histidine-HCl. In some embodiments, the composition comprises total histidine (e.g., histidine plus histidine-HCl) at a concentration of about 50-75 mM. In some embodiments, the composition comprises total histidine at a concentration of about 50 mM. In some embodiments, the tonicity agent is arginine-hydrochloride (HCl). In some embodiments, the composition comprises arginine-HCl at a concentration of about 100 mM.

In some embodiments, the stabilizer is a carbohydrate. In some embodiments, the stabilizer is a hexose. In some embodiments, the stabilizer is a trehalose. In some embodiments, the composition comprises stabilizer at a concentration of about 50-100 mg/ml. In some embodiments, the composition comprises stabilizer at a concentration of about 50 mg/ml. In some embodiments, the stabilizer is sucrose. In some embodiments, the composition comprises sucrose at a concentration of about 50 mg/ml.

In some embodiments, the surfactant is polysorbate 80 (PS80). In some embodiments, the composition comprises PS80 at a concentration of about 0.1-0.6 mg/ml. In some embodiments, the composition comprises PS80 at a concentration of about 0.2 mg/ml. In some embodiments, the composition has a pH of about 6.0-7.0. In some embodiments, the composition has a pH of about 6.5-6.7. In some embodiments, the composition has a pH of about 6.6.

In some aspects, disclosed herein is a composition comprising 10 mg/ml of a CD38-binding fusion protein, 50 mM of histidine, 100 mM of arginine, 50 mg/ml of sucrose, and 0.2 mg/ml of polysorbate 80 (PS80), wherein the composition has a pH of 6.6, wherein the CD38-binding fusion protein comprises an anti-CD38 antibody comprising a heavy chain and a light chain, wherein the heavy chain comprises the amino acid sequence of SEQ ID NO: 9 and is fused to an attenuated interferon alpha-2b comprising the amino acid sequence of SEQ ID NO: 12, and wherein the light chain comprises the amino acid sequence of SEQ ID NO: 10.

In some embodiments, described herein is a composition comprising 30-100 mg/ml of a CD38-binding fusion protein, 50-75 mM of histidine, 100-150 mM of arginine, 50-100 mg/ml of sucrose, 0.1 to 0.6 mg/ml of polysorbate 80 (PS80), wherein the composition has a pH of 6.0-7.0, wherein the CD38-binding fusion protein comprises an anti-CD38 antibody comprising a heavy chain and a light chain, wherein the heavy chain comprises the amino acid sequence of SEQ ID NO: 9 and is fused to an attenuated interferon alpha-2b comprising the amino acid sequence of SEQ ID NO: 12, and wherein the light chain comprises the amino acid sequence of SEQ ID NO: 10. In some embodiments, the composition comprises 40 mg/ml of the CD38 binding fusion protein. In some embodiments, the composition comprises 60 mg/ml of the CD38 binding fusion protein. In some embodiments, the composition comprises 80 mg/ml of the CD38 binding fusion protein.

In some embodiments, the CD38-binding fusion protein comprises a heavy chain comprising the amino acid sequence of SEQ ID NO: 13 and a light chain comprising the amino acid sequence of SEQ ID NO: 10. In some embodiments, the composition is lyophilized. In some embodiments, the composition is in dosage unit form.

In some aspects, this application discloses a method of treating a CD38-expressing cancer, the method comprising administering to a subject in need thereof an effective amount of a composition described herein. In some embodiments, the CD38-expressing cancer is B-cell lymphoma, multiple myeloma, Waldenstrom's macroglobulinemia, non-Hodgkin's lymphoma, chronic myelogenous leukemia, chronic lymphocytic leukemia, or acute lymphocytic leukemia. In some embodiments, the CD38-expressing cancer is multiple myeloma. In some embodiments, the multiple myeloma is refractory multiple myeloma. In some embodiments, the subject is human.

In some embodiments, the method further comprises administering to the subject lenalidomide or pomalidomide. In some embodiments, the composition is for use in a method for treating a CD38-expressing cancer in a subject. In some embodiments, the subject is receiving treatment with lenalidomide or pomalidomide.

In some embodiments, the method described herein, further comprises administering to the subject a CD47 antagonist. In some embodiments, the subject is receiving treatment with a CD47 antagonist.

### BRIEF DESCRIPTION OF DRAWINGS

The drawings are for illustration purposes only, not for limitation.

FIGS. 1A-1F show the effects of solution pH on CD38-binding fusion protein composition quality attributes. pH 5.2, 5.51, 5.93, 6.37, 6.8 and 7.23 were tested. The CD38-binding fusion protein compositions had 1 mg/ml CD38-binding fusion protein, 2.3 mM citrate, 5 mM sodium phosphate (phosphate-Na) buffer, and 50 mM sodium chloride (NaCl). Measurements were taken upon making the composition, after 2 weeks at 25° C., and after 2 weeks at 40° C. FIG. 1A-1C measured the fraction of CD38-binding fusion protein in the major, acidic and basic peaks using cation exchange chromatography (CEX). Results show pH 6.37 and pH 6.8 have similar fraction of the major, acidic and basic peaks compared to the other pHs tested. FIG. 1D measured aggregation in the CD38-binding fusion protein composition using size exclusion chromatograph (SEC). Results showed that the amount of aggregation remains approximately at the initial and 2 week 25° C. timepoint regardless of pH. However, increased aggregation was observed starting at pH 6.37 at the 40° C. 2 week time point. FIG. 1E shows that the amount of low molecular weight molecules (LMW) (as measured by SEC) has little to no change across all pH's, times and temperatures tested. FIG. 1F shows that the amount of CD38-binding fusion protein in the composition has little to no change across all pH's tested with a slight decline in the 40° C. 2 week condition at pH 6.8 and 7.23.

FIG. 2 shows the effect of pH on CD38-binding fusion protein stability. The melting temperature of the CD38-binding fusion protein in pH 5.1, 6.5 and 10 was measured using calorimetry. The CD38-binding fusion protein solutions at pH 5.1 and 6.5 had a composition of 1 mg/ml CD38-binding fusion protein, 2.34 mM Citrate, 5 mM Phosphate-Na buffer, and 50 mM NaCl. The CD38-binding fusion protein solution at pH 10 had a composition of 1 mg/ml C38-binding fusion protein, 0.234 mM Citrate, 0.5 mM Phosphate-Na buffer, 5.0 mM NaCl. Results suggested that increasing pH had a minor parabolic effect on melting temperature: pH 5.1 (55.69° C.), pH 6.5 (58.02° C.), and pH 10 (57.83° C.).

FIG. 3 shows that increasing histidine buffer concentration and pH in the composition comprising the CD38-binding fusion protein decreases (improves) composition turbidity (defined in illustration as appearance), decreases the concentration of the basic CD38-binding fusion protein and increases the concentration of the acidic CD38-binding fusion protein. Compositions tested are listed in Table 3. FIG. 3 left panel shows that increasing histidine buffer concentration from 12.5 mM to 107.5 mM decreases the composition turbidity, decreases the concentration of the CD38-binding fusion protein basic peak in the composition, increases the concentration of the CD38-binding fusion protein acidic peak in the composition, and has negligible effects on CD38-binding fusion protein aggregation and dimerization. FIG. 3 center column shows that increasing the CD38-binding fusion protein to polysorbate-80 molar ratio from 0.5 to 2 slightly decreases formulation turbidity, slightly increases the concentration of the CD38-binding fusion protein basic peak in the composition, slightly decreases the concentration of the CD38-binding fusion protein acidic peak in the composition, and has negligible effects on CD38-binding fusion protein aggregation and dimerization. FIG. 3 right column shows that increasing pH from 5.5 to 6.5 decreases formulation turbidity, decreases the concentration of the CD38-binding fusion protein basic peak in the composition, increases the concentration of the CD38-binding fusion protein acidic peak in the composition, and has negligible effects on CD38-binding fusion protein aggregation and dimerization.

FIG. 4 shows the effects of altering the protein concentration, histidine concentration, pH, and the polysorbate-80 to CD38-binding fusion protein ratio of the composition. Composition subvisible particle size, subvisible particle concentration, turbidity (defined in illustration as appearance), concentration of the CD38-binding fusion protein main (major) peak, concentration of the CD38-binding fusion protein basic peak, concentration of the CD38-binding fusion protein acidic peak, and CD38-binding fusion protein aggregation were measured. Compositions tested are listed in Table 5. FIG. 4 the bottom three rows and top row show that increasing CD38-binding fusion protein concentration (5-25 mg/ml), histidine buffer concentration (40-60 mM), pH (6.2-7.1), and polysorbate-80/CD38-binding fusion protein molar ratio (0.5-1.5) of the composition has little to no effect on subvisible particle concentration or aggregation. FIG. 4 far left column shows that increasing CD38-binding fusion protein concentration (5-25 mg/ml) has little to no effect on the CD38-binding fusion protein main peak, the CD38-binding fusion protein basic peak, the CD38-binding fusion protein acidic peak, and CD38-binding fusion protein aggregation. FIG. 4 middle left column shows that increasing histidine buffer concentration (40-60 mM) of the composition has little to no effect on the main CD38-binding fusion protein peak, the CD38-binding fusion protein basic peak, the CD38-binding fusion protein acidic peak, and CD38-binding fusion protein aggregation. FIG. 4 center right column shows that increasing pH (6.2-71) of the composition slightly increases then slightly decreases the concentration of CD38-binding fusion protein main peak, decreases the concentration of the CD38-binding fusion protein basic peak, increases the concentration of the CD38-binding fusion protein acidic peak, and has a negligible effect on CD38-binding fusion protein aggregation. FIG. 4 far right column shows that increasing the polysorbate-80/CD38-binding fusion protein molar ratio (0.5 to 1.5) of the composition has little to no effect on the concentration of CD38-binding fusion protein main peak, the concentration of the CD38-binding fusion protein basic peak, the concentration of the CD38-binding fusion protein acidic peak, and on CD38-binding fusion protein aggregation.

FIG. 5 shows that changing the concentration of histidine, histidine-HCl and arginine-HCl by +/−2% has a minimal effect on the pH of a CD38-binding fusion protein composition. The base composition comprised all of the following components with varying amounts of the component that was being changes: 10 mg/ml CD38-binding fusion protein containing sequences as provided in Table 1, 50 mM histidine, 5% sucrose, 100 mM Arg-HCl, 0.007% polysorbate-80 and a pH of 6.6. The concentration of histidine, histidine-HCl and arginine-HCl were each altered individually.

FIG. 6 shows the potency of the CD38-binding fusion protein composition over time after storage at about 5° C. Each CD38-binding fusion protein composition lot comprised: 10 mg/ml of a CD38-binding fusion protein comprising SEQ ID NOs: 1-13, 50 mM histidine, 5% w/v sucrose, 100 mM Arginine-HCl, 0.02% PS80, and a pH of 6.6. Lyophilized compositions were stored at 5° C.±3° C. Results show that storage for up to 12 months at 5° C.±3° C. decreasing potency by at most about 10%.

### DETAILED DESCRIPTION

Various terms relating to aspects of disclosure are used throughout the specification and claims. Such terms are to be given their ordinary meaning in the art, unless otherwise indicated. Other specifically defined terms are to be construed in a manner consistent with the definition provided herein.

As used herein, the singular forms “a,” “an,” and “the” include plural referents unless expressly stated otherwise.

The present disclosure, in some aspects, relates to compositions comprising a CD38-binding fusion protein, wherein the CD38-binding fusion comprises an anti-CD38 antibody fused to an attenuated interferon alpha-2b protein. In some embodiments, a composition comprising a CD38-binding fusion protein described herein further comprises a buffer (e.g., a histidine/histidine-HCl buffer), a tonicity agent (e.g., arginine-HCl), a stabilizer (e.g., sucrose), and a surfactant (e.g., polysorbate such as polysorbate 80). In some embodiments, a composition described herein has a pH between 6.0-7.0 (e.g., 6.6) and comprises a CD38-binding fusion protein at a concentration of 8-12 mg/ml (e.g., 10 mg/ml), histidine/histidine-HCl at a concentration of 40-60 mM (e.g., 50 mM), arginine-HCl at a concentration of 75-125 mM (e.g., 100 mM), sucrose at a concentration of 30-80 mg/ml (e.g., 50 mg/ml), and polysorbate 80 at a 0.1-0.3 mg/ml (e.g., 0.2 mg/ml). In some embodiments, a CD38-binding fusion protein described herein is stable and/or remains active in the composition (e.g., when stored for periods of months to years). In some embodiments, a composition described herein is an aqueous solution. In some embodiments, a composition described herein is in lyophilized form. Methods of using the compositions described herein for treating cancer are also provided.

A “CD38-binding fusion protein,” as used herein, refers to a fusion protein comprising a CD38 binding domain fused to an attenuated interferon alpha-2b protein. A “fusion protein” refers to a polypeptide comprising two or more proteinaceous components associated by at least one covalent bond which is a peptide bond, regardless of whether the peptide bond involves the participation of a carbon atom of a carboxyl acid group or involves another carbon atom. The term “fuse” refers to the act of creating a fused molecule as described above, such as, e.g., a fusion protein generated from the recombinant fusion of genetic regions which when translated produces a single proteinaceous molecule. CD38-binding fusion proteins that may be used in the compositions described herein are described in the art, e.g., in U.S. Pat. No. 10,544,199B2, incorporated herein by reference. The amino acid sequences of an example of an anti-CD38antibody are provided in Table 1.

A CD38-binding fusion protein in a composition described herein comprises an anti-CD38 antibody. The term “antibody,” as used herein includes, for example, an intact immunoglobulin or an antigen binding portion of an immunoglobulin or an antigen binding protein related or derived from an immunoglobulin. Intact antibody structural units often comprise a tetrameric protein. Each tetramer is typically composed of two identical pairs of polypeptide chains, each pair having one “light” chain (typically having a molecular weight of about 25 kDa) and one “heavy” chain (typically having a molecular weight of about 50-to 70 kDa). Human immunoglobulin light chains may be classified as having kappa or lambda light chains. In some embodiments, the antibodies described herein comprise antigen binding domains (e.g., antibody heavy and/or light chains) that generally are based on the IgG class, which has several subclasses, including, but not limited to IgG1, IgG2, IgG3, and IgG4. In general, IgG1 has different allotypes with polymorphisms at 356 (D or E), IgG2 and 358 (L or M). The sequences depicted herein use the 356D/358M allotype; however any allotype is included herein and can be used in accordance with the present disclosure. For example, any sequence inclusive of an IgG1 Fc domain included herein can have 356E/358L replacing the 356D/358M allotype.

The anti-CD38 antibody of the CD38-binding fusion protein in the compositions described herein comprise a heavy chain comprising a heavy chain variable domain (VH) and a light chain comprising a light chain variable domain (VL). A “variable domain,” as used herein, refers to the region of an immunoglobulin that comprises one or more Ig domains substantially encoded by any of the Vκ(V.kappa), Vλ (V.lamda), and/or VH genes that make up the kappa, lambda, and heavy chain immunoglobulin genetic loci respectively. In the variable domains, three loops are gathered for each of the V domains of the heavy chain and light chain to form an antigen-binding site. Each of the loops is referred to as a complementarity-determining region (hereinafter referred to as a “CDR”). Additionally, the variable domains also contain relatively invariant stretches called framework regions (FRs) of 15-30 amino acids separated by CDRs. Each VH and VL is composed of three CDRs and four FRs, arranged from amino-terminus to carboxy-terminus in the following order: FR1-CDR1-FR2-CDR2-FR3-CDR3-FR4. In some embodiments, an “antibody molecule” refers to two-chain and multi-chain immunoglobulin proteins and glycoproteins. In some embodiments, an anti-CD38 antibody of the CD38-binding fusion protein in the compositions described herein is an antibody fragment or antigen binding fragment of an antibody, including, for example, Fab, Fab′, F(ab′)2, and Fv fragments.

In some embodiments, an anti-CD38 antibody of the CD38-binding fusion protein in the compositions described herein comprises a VH comprising a CDRH1 comprising the amino acid sequence of SEQ ID NO: 1, a CDRH2 comprising the amino acid sequence of SEQ ID NO: 2, and a CDRH3 comprising the amino acid sequence of SEQ ID NO: 3; and a VL comprising a CDRL1 comprising the amino acid sequence of SEQ ID NO: 4, a CDRL2 comprising the amino acid sequence of SEQ ID NO: 5, and a CDRL3 comprising the amino acid sequence of SEQ ID NO: 6. In some embodiments, an anti-CD38 antibody of the CD38-binding fusion protein in the compositions described herein comprises a set of 6 CDRs that collectively contain up to 10 (e.g., 1, 2, 3, 4, 5, 6, 7, 8, 9, or 10) amino acid modifications, relative to the 6 CDRs of the anti-CD38 antibody provided in Table 1. For example, in some embodiments, the CDRs can be modified in any fashion, as long as the total number of changes in the set of 6 CDRs does not exceed 10 amino acid modifications, with any combination of CDRs being changed; e.g., there may be one change in CDRL1, two in CDRH2, none in CDRH3, etc. In some embodiments, each CDR has no more than a single amino acid substitution relative to the corresponding CDR of the anti-CD38 antibody provided in Table 1. In some embodiments, amino acid modifications in the CDRH3 are avoided.

In some embodiments, an anti-CD38 antibody of the CD38-binding fusion protein in a composition described herein comprises a VH comprising the amino acid sequence of SEQ ID NO: 7 and a VL comprising the amino acid sequence of SEQ ID NO: 8. In some embodiments, an anti-CD38 antibody of the CD38-binding fusion protein in a composition described herein comprises a VH comprising an amino acid sequence that is at least 80% (e.g., at least 80%, at least 85%, at least 90%, at least 95%, at least 99%) identical to the amino acid sequence of SEQ ID NO: 7 and a VL comprising an amino acid sequence that is at least 80% (e.g., at least 80%, at least 85%, at least 90%, at least 95%, at least 99%) identical to the amino acid sequence of SEQ ID NO: 8.

In some embodiments, an anti-CD38 antibody of the CD38-binding fusion protein in a composition described herein is a full-length IgG antibody. In a full-length IgG antibody, each heavy chain is comprised of a heavy chain variable region (abbreviated herein as VH) and a heavy chain constant region. Each light chain is comprised of a light chain variable region (abbreviated herein as VL) and a light chain constant region. In some embodiments, the Immunoglobulin molecules are IgG class IgG4, or a subclass thereof.

In some embodiments, an anti-CD38 antibody of the CD38-binding fusion protein in a composition described herein comprises an IgG4 constant region (e.g., a human IgG4 constant region comprising the amino acid sequence of SEQ ID NO: 14). As used herein, the term “IgG4 constant region” refers to a wild-type IgG4 constant region (e.g., a wild-type human IgG4 constant region) or an IgG4 constant region variant (e.g., a human IgG4 constant region variant) or fragment thereof. IgG4 constant region variants (e.g., human IgG4 constant region variants) that may be used in the anti-CD38 antibody of the CD38-binding fusion protein in a composition described herein may, in some embodiments, comprise one or more mutations, e.g., mutations that stabilize the hinge region and/or reduce the toxicity of the antibody. For example, a mutation at position 228 of the IgG4 according to the EU numbering system stabilizes the hinge of IgG4. In some embodiments, a mutation at position 228 of the IgG4 constant region according to the EU numbering system results in a proline at position 228.

In some embodiments, mutations in the IgG4 constant region decrease antibody dependent cell cytotoxicity (ADCC). “Antibody dependent cell-mediated cytotoxicity (ADCC),” as used herein, refers to a cell-mediated reaction wherein nonspecific cytotoxic cells that express Fc gamma receptors (FcγRs) recognize bound antibody on a target cell and subsequently cause lysis of the target cell. In some embodiments, an anti-CD38 antibody of the CD38-binding fusion protein in a composition described herein comprises an IgG4 constant region comprising one or more mutations that reduce ADCC to avoid undesirably high levels of cytotoxicity (e.g., mutations at one or more of positions 252, 254, and 256 of the IgG4 constant region according to the EU numbering system). In some embodiments, a mutation at position 252 of the IgG4 constant region according to the EU numbering system results in a tyrosine at position 252. In some embodiments, a mutation at position 254 of the IgG4 constant region according to the EU numbering system results in a threonine at position 254. In some embodiments, a mutation at position 256 of the IgG4 constant region according to the EU numbering system results in a glutamic acid at position 256.

In some embodiments, an anti-CD38 antibody of the CD38-binding fusion protein in a composition described herein comprises an IgG4 constant region comprising a mutation at position 228 of the IgG4 constant region according to the EU numbering system. In some embodiments, an anti-CD38 antibody of the CD38-binding fusion protein in a composition described herein comprises an IgG4 constant region comprising the amino acid sequence of SEQ ID NO: 15.

In some embodiments, an anti-CD38 antibody of the CD38-binding fusion protein in a composition described herein comprises a heavy chain comprising a VH and a human IgG4 constant region, wherein the VH comprises the amino acid sequence of SEQ ID NO: 7 and the IgG4 constant region comprises the amino acid sequence of SEQ ID NO: 15. In some embodiments, an anti-CD38 antibody of the CD38-binding fusion protein in a composition described herein comprises a heavy chain comprising the amino acid sequence of SEQ ID NO: 9. In some embodiments, an anti-CD38 antibody of the CD38-binding fusion protein in a composition described herein comprises a heavy chain comprising an amino acid sequence at least 80% (e.g., at least 80%, at least 85%, at least 90%, at least 95%, at least 99%) identical to the amino acid sequence of SEQ ID NO: 9.

In some embodiments, an anti-CD38 antibody of the CD38-binding fusion protein in a composition described herein comprises a light chain comprising a VL and a kappa light constant region, wherein the VL comprises the amino acid sequence of SEQ ID NO: 8. In some embodiments, an anti-CD38 antibody of the CD38-binding fusion protein in a composition described herein comprises a light chain comprising the amino acid sequence of SEQ ID NO: 10. In some embodiments, an anti-CD38 antibody of the CD38-binding fusion protein in a composition described herein comprises a light chain comprising an amino acid sequence at least 80% (e.g., at least 80%, at least 85%, at least 90%, at least 95%, at least 99%) identical to the amino acid sequence of SEQ ID NO: 10.

In some embodiments, a CD38-binding fusion protein in a composition described herein further comprises an anti-CD38 antibody (e.g., the anti-CD38 antibody provided in Table 1) fused to an attenuated interferon alpha-2b protein (e.g., the attenuated interferon alpha-2b protein is fused to the heavy chain of the anti-CD38 antibody). It has been observed that interferon-alpha-2b can be attenuated in its biologic activity, which is mediated through the interferon binding to an interferon receptor on a cell surface, by introducing certain amino acid changes into the protein sequence. In some embodiments, an attenuated interferon alpha-2b protein comprises mutations that reduce its potency (e.g., A145D) and/or eliminate O-linked glycosylation of the interferon alpha-2b protein (e.g., T106A). An attenuated interferon molecule can be fused to antibodies that specifically bind to CD38 (e.g., an anti-CD38 antibody), as described herein, such that the anti-CD38 antibody may serve as a delivery vehicle for the attenuated interferon to CD38-positive cells with a resulting diminution of off target interferon activity caused by the attenuated interferon molecule.

In some embodiments, the attenuated interferon alpha-2b protein is fused to the heavy chain of the anti-CD38 antibody. In some embodiments, the attenuated interferon alpha-2b protein is fused to the C-terminus of the heavy chain of the anti-CD38 antibody. As such, in some embodiments, the CD38-binding fusion protein in a composition described herein comprises a heavy chain and a light chain, wherein the heavy chain comprises the heavy chain of an anti-CD38 antibody fused to an attenuated interferon alpha-2b protein and wherein the light chain is the light chain of the anti-CD38 antibody. In some embodiments, the CD38-binding fusion protein in a composition described herein comprises two heavy chains and two light chains, wherein each heavy chain comprises the heavy chain of an anti-CD38 antibody fused to an attenuated interferon alpha-2b protein and wherein each light chain is the light chain of the anti-CD38 antibody.

In some embodiments, the attenuated interferon alpha-2b comprises T106A and A145D mutations relative to a wild type human interferon alpha-2b (e.g., a human interferon alpha-2b comprising the amino acid sequence of SEQ ID NO: 11). In some embodiments, the attenuated interferon alpha-2b comprises the amino acid of SEQ ID NO: 12. In some embodiments, the attenuated interferon alpha-2b comprises an amino acid sequence at least 80% (e.g., at least 80%, at least 85%, at least 90%, at least 95%, at least 99%) identical to the amino acid of SEQ ID NO: 12.

In some embodiments, a CD38-binding fusion protein in a composition described herein comprises a heavy chain comprising an amino acid sequence at least 80% (e.g., at least 80%, at least 85%, at least 90%, at least 95%, at least 99%) identical to the amino acid of SEQ ID NO: 13 and a light chain comprising an amino acid sequence at least 80% (e.g., at least 80%, at least 85%, at least 90%, at least 95%, at least 99%) identical to the amino acid of SEQ ID NO: 10. In some embodiments, a CD38-binding fusion protein in a composition described herein comprises the amino acid of SEQ ID NO: 13 and a light chain comprising the amino acid of SEQ ID NO: 10. In some embodiments, a CD38-binding fusion protein in a composition described herein comprises two heavy chains and two light chains, wherein each heavy comprises the amino acid sequence of SEQ ID NO: 13 and each light chain comprises the amino acid sequence of SEQ ID No: 10.

TABLE 1
anti-CD-38 fusion protein amino acid sequences

| | | | SEQ ID |
|---|---|---|---|
| | | Sequence | NO |
| anti-CD38 | CDR-H1 | DSVMN | 1 |
| antibody | CDR-H2 | WIDPEYGRTDVAEKFQG | 2 |
| *CDRs are | CDR-H3 | TKYNSGYGFPY | 3 |
| according | CDR-L1 | KASQNVDSDVD | 4 |
| to Kabat | CDR-L2 | KASNDYT | 5 |
| numbering | CDR-L3 | MQSNTHPRT | 6 |
| | VH | EVQLVQSGAEVKKPGATVKISCKVSGYTFTDSV | 7 |
| | | MNWVQQAPGKGLEWMGWIDPEYGRTDVAEKF | |
| | | QGRVTITADTSTDTAYMELSSLRSEDTAVYYCAR | |
| | | TKYNSGYGFPYWGQGTTVTVSS | |
| | VL | DIQMTQSPSSLSASVGDRVTITCKASQNVDSDVD | 8 |
| | | WYQQKPGKAPKLLIYKASNDYTGVPSRFSGSGS | |
| | | GTDFTFTISSLQPEDIATYYCMQSNTHPRTFGGGT | |
| | | KVEIKR | |
| | HC | EVQLVQSGAEVKKPGATVKISCKVSGYTFTDSV | 9 |
| | | MNWVQQAPGKGLEWMGWIDPEYGRTDVAEKF | |
| | | QGRVTITADTSTDTAYMELSSLRSEDTAVYYCAR | |
| | | TKYNSGYGFPYWGQGTTVTVSSASTKGPSVFPL | |
| | | APCSRSTSESTAALGCLVKDYFPEPVTVSWNSGA | |
| | | LTSGVHTFPAVLQSSGLYSLSSVVTVPSSSLGTKT | |
| | | YTCNVDHKPSNTKVDKRVESKYGPPCPPCPAPEF | |
| | | LGGPSVFLFPPKPKDTLMISRTPEVTCVVVDVSQ | |
| | | EDPEVQFNWYVDGVEVHNAKTKPREEQFNSTYR | |
| | | VVSVLTVLHQDWLNGKEYKCKVSNKGLPSSIEK | |
| | | TISKAKGQPREPQVYTLPPSQEEMTKNQVSLTCL | |
| | | VKGFYPSDIAVEWESNGQPENNYKTTPPVLDSD | |
| | | GSFFLYSRLTVDKSRWQEGNVFSCSVMHEALHN | |
| | | HYTQKSLSLSLGK | |
| | LC | DIQMTQSPSSLSASVGDRVTITCKASQNVDSDVD | 10 |
| | | WYQQKPGKAPKLLIYKASNDYTGVPSRFSGSGS | |

| | | |
|---|---|---|
| | GTDFTFTISSLQPEDIATYYCMQSNTHPRTFGGGT | |
| | KVEIKRTVAAPSVFIFPPSDEQLKSGTASVVCLLN | |
| | NFYPREAKVQWKVDNALQSGNSQESVTEQDSK | |
| | DSTYSLSSTLTLSKADYEKHKVYACEVTHQGLSS | |
| | PVTKSFNRGEC | |
| WT interferon alpha-2b | CDLPQTHSLGSRRTLMLLAQMRRISLFSCLKDRH | 11 |
| | DFGFPQEEFGNQFQKAETIPVLHEMIQQIFNLFST | |
| | KDSSAAWDETLLDKFYTELYQQLNDLEACVIQG | |
| | VGVTETPLMKEDSILAVRKYFQRITLYLKEKKYS | |
| | PCAWEVVRAEIMRSFSLSTNLQESLRSKE | |
| Attenuated interferon alpha-2b | CDLPQTHSLGSRRTLMLLAQMRRISLFSCLKDRH | 12 |
| (T106A, A145D) | DFGFPQEEFGNQFQKAETIPVLHEMIQQIFNLFST | |
| | KDSSAAWDETLLDKFYTELYQQLNDLEACVIQG | |
| | VGVAETPLMKEDSILAVRKYFQRITLYLKEKKYS | |
| | PCAWEVVRDEIMRSFSLSTNLQESLRSKE | |
| HC of anti-CD38 antibody | EVQLVQSGAEVKKPGATVKISCKVSGYTFTDSV | 13 |
| fused to Attenuated interferon | MNWVQQAPGKGLEWMGWIDPEYGRTDVAEKF | |
| alpha-2b (T106A, A145D) | QGRVTITADTSTDTAYMELSSLRSEDTAVYYCAR | |
| | TKYNSGYGFPYWGQGTTVTVSSASTKGPSVFPL | |
| | APCSRSTSESTAALGCLVKDYFPEPVTVSWNSGA | |
| | LTSGVHTFPAVLQSSGLYSLSSVVTVPSSSLGTKT | |
| | YTCNVDHKPSNTKVDKRVESKYGPPCPPCPAPEF | |
| | LGGPSVFLFPPKPKDTLMISRTPEVTCVVVDVSQ | |
| | EDPEVQFNWYVDGVEVHNAKTKPREEQFNSTYR | |
| | VVSVLTVLHQDWLNGKEYKCKVSNKGLPSSIEK | |
| | TISKAKGQPREPQVYTLPPSQEEMTKNQVSLTCL | |
| | VKGFYPSDIAVEWESNGQPENNYKTTPPVLDSD | |
| | GSFFLYSRLTVDKSRWQEGNVFSCSVMHEALHN | |
| | HYTQKSLSLSLGKCDLPQTHSLGSRRTLMLLAQMR | |
| | RISLFSCLKDRHDFGFPQEEFGNQFQKAETIPVLHE | |
| | MIQQIFNLFSTKDSSAAWDETLLDKFYTELYQQLND | |
| | LEACVIQGVGVAETPLMKEDSILAVRKYFQRITLYLK | |
| | EKKYSPCAWEVVRDEIMRSFSLSTNLQESLRSKE | |
| Wild type human IgG4 | ASTKGPSVFPLAPCSRSTSESTAALGCLVKDYFPE | 14 |
| constant region | PVTVSWNSGALTSGVHTFPAVLQSSGLYSLSSVV | |
| | TVPSSSLGTKTYTCNVDHKPSNTKVDKRVESKY | |
| | GPPCPSCPAPEFLGGPSVFLFPPKPKDTLMISRTPE | |
| | VTCVVVDVSQEDPEVQFNWYVDGVEVHNAKTK | |
| | PREEQFNSTYRVVSVLTVLHQDWLNGKEYKCKV | |
| | SNKGLPSSIEKTISKAKGQPREPQVYTLPPSQEEM | |
| | TKNQVSLTCLVKGFYPSDIAVEWESNGQPENNY | |
| | KTTPPVLDSDGSFFLYSRLTVDKSRWQEGNVFSC | |
| | SVMHEALHNHYTQKSLSLSLGK | |
| Human IgG4 constant region | ASTKGPSVFPLAPCSRSTSESTAALGCLVKDYFPE | 15 |
| variant with S228P mutation | PVTVSWNSGALTSGVHTFPAVLQSSGLYSLSSVV | |
| (EU numbering system) | TVPSSSLGTKTYTCNVDHKPSNTKVDKRVESKY | |
| | GPPCPPCPAPEFLGGPSVFLFPPKPKDTLMISRTPE | |
| | VTCVVVDVSQEDPEVQFNWYVDGVEVHNAKTK | |
| | PREEQFNSTYRVVSVLTVLHQDWLNGKEYKCKV | |
| | SNKGLPSSIEKTISKAKGQPREPQVYTLPPSQEEM | |
| | TKNQVSLTCLVKGFYPSDIAVEWESNGQPENNY | |
| | KTTPPVLDSDGSFFLYSRLTVDKSRWQEGNVFSC | |
| | SVMHEALHNHYTQKSLSLSLGK | |

In some embodiments, a composition described herein comprises a CD38-binding fusion protein at a concentration that does not exceed 100 mg/ml. In some embodiments, a composition described herein comprises a CD38-binding fusion protein at a concentration of 1-100 mg/ml, 5-100 mg/ml, 10-100 mg/ml, 20-100 mg/ml, 30-100 mg/ml, 40-100 mg/ml, 50-100 mg/ml, 60-100 mg/ml, 70-100 mg/ml, 30-80 mg/ml, 40-80 mg/ml, or 40-60 mg/ml. In some embodiments, a composition described herein comprises a CD38-binding fusion protein at a concentration of 25-35 mg/ml, 27.5-32.5 mg/ml, 29-31 mg/ml, or 29.5-30.5 mg/ml. In some embodiments, a composition described herein comprises a CD38-binding fusion protein at a concentration of 35-45 mg/ml, 37.5-42.5 mg/ml, 39-41 mg/ml, or 39.5-40.5 mg/ml. In some embodiments, a composition described herein comprises a CD38-binding fusion protein at a concentration of 55-65 mg/ml, 57.5-62.5 mg/ml, 59-61 mg/ml, or 59.5-60.5 mg/ml. In some embodiments, a composition described herein comprises a CD38-binding fusion protein at a concentration of 75-85 mg/ml, 77.5-82.5 mg/ml, 79-61 mg/ml, or 79.5-80.5 mg/ml. In some embodiments, a composition described herein comprises a CD38-binding fusion protein at a concentration of 30 mg/ml. In some embodiments, a composition described herein comprises a CD38-binding fusion protein at a concentration of 40 mg/ml. In some embodiments, a composition described herein comprises a CD38-binding fusion protein at a concentration of 50 mg/ml. In some embodiments, a composition described herein comprises a CD38-binding fusion protein at a concentration of 60 mg/ml. In some embodiments, a composition described herein comprises a CD38-binding fusion protein at a concentration of 80 mg/ml. In some embodiments, a composition described herein comprises a CD38-binding fusion protein at a concentration of 100 mg/ml. In some embodiments, a composition described herein comprises a CD38-binding fusion protein at a concentration of 8-12 mg/ml. For example, a composition described herein may comprise a CD38-binding fusion protein at a concentration of 8-12 mg/ml, 8-11.5 mg/ml, 8-11 mg/ml, 8-10.5 mg/ml, 8-10 mg/ml, 8-9.5 mg/ml, 8-9 mg/ml, 8-8.5 mg/ml, 8.5-12 mg/ml, 8.5-11.5 mg/ml, 8.5-11 mg/ml, 8.5-10.5 mg/ml, 8.5-10 mg/ml, 8.5-9.5 mg/ml, 8.5-9 mg/ml, 9-12 mg/ml, 9-11.5 mg/ml, 9-11 mg/ml, 9-10.5 mg/ml, 9-10 mg/ml, 9-9.5 mg/ml, 9.5-12 mg/ml, 9.5-11.5 mg/ml, 9.5-11 mg/ml, 9.5-10.5 mg/ml, 9.5-10 mg/ml, 10-12 mg/ml, 10-11.5 mg/ml, 10-11 mg/ml, 10-10.5 mg/ml, 10.5-12 mg/ml, 10.5-11.5 mg/ml, 10.5-11 mg/ml, 11-12 mg/ml, 11-11.5 mg/ml, or 11.5-12 mg/ml. In some embodiments, a composition described herein comprises a CD38-binding fusion protein at a concentration of about 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, 11, 11.1, 11.2, 11.3, 11.4, 11.5, 11.6, 11.7, 11.8, 11.9, or 12 mg/ml. In some embodiments, a composition described herein comprises a CD38-binding fusion protein at a concentration of about 10 mg/ml.

In some embodiments, a composition described herein has a pH of 5.5-7.5. For example, a composition described herein may have a pH of 5.5-7.5, 5.5-7, 5.5-6.5, 5.5-6, 6-7.5, 6.0-7.0, 6-6.5, 6.5-7.5, 6.5-7, or 7-7.5. In some embodiments, a composition described herein has a pH of about 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, or 7.5. In some embodiments, a composition described herein has a pH of about 6.1-7.1 (e.g., 6.1-7.1, 6.2-7, 6.3-6.9, 6.4-6.8, or 6.5-6.7). In some embodiments, a composition described herein has a pH of about 6.6.

A composition as described herein further comprises a buffer (e.g., a histidine/histidine-HCl buffer), a tonicity agent (e.g., arginine-HCl), a stabilizer (e.g., sucrose), and a surfactant (e.g., polysorbate such as polysorbate 80). The buffer may also have stabilizing properties. The tonicity agent may also have stabilizing properties. The surfactant may also have stabilizing properties.

In some embodiments, a composition described herein comprises a buffer comprising histidine and histidine-HCl. In some embodiments, the histidine and histidine-HCl balance results in a final histidine concentration in the composition of 10-120 mM (e.g., 10-120 mM, 20-110 mM, 30-100 mM, 40-90 mM, 50-80 mM, or 60-70 mM). In some embodiments, the histidine and histidine-HCl balance results in a final histidine concentration in the composition of 12.5-107.5 mM. In some embodiments, the histidine and histidine-HCl balance results in a final histidine concentration in the composition of about 15-75 mM (e.g., 15 mM, 20 mM, 25 mM, 30 mM, 35 mM, 40 mM, 45 mM, 50 mM, 55 mM, 60 mM, 65 mM, 70 mM, or 75 mM). In some embodiments, the histidine and histidine-HCl balance results in a final histidine concentration in the composition of 50-75 mM. In some embodiments, the histidine and histidine-HCl balance results in a final histidine concentration in the composition of 15-50 mM (e.g., about 15 mM, about 20 mM, about 25 mM, about 30 mM, about 35 mM, about 40 mM, about 45 mM, or about 50 mM). In some embodiments, the histidine and histidine-HCl balance results in a final histidine concentration in the composition of about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 mM of histidine.

The relative amount of histidine and histidine-HCl may be adjusted, e.g., to achieve a desired pH, while maintaining the histidine concentration in the composition, as described herein. In some embodiments, the histidine and histidine-HCl balance results in a final histidine concentration in the composition of about 15 mM (e.g., when the composition comprises a buffer comprising histidine at a concentration of 7.5 mM and histidine-HCl at a concentration of 7.5 mM). In some embodiments, the histidine and histidine-HCl balance results in a final histidine concentration in the composition of about 50 mM (e.g., when the composition comprises a buffer comprising histidine at a concentration of 40 mM and histidine-HCl at a concentration of 10 mM).

In some embodiments, a composition described herein comprises a tonicity agent comprising arginine (e.g., arginine-HCl). In some embodiments, a composition described herein comprises arginine at a concentration of 50-150 mM (e.g., 50-125 mM, 60-120 mM, 70-110 mM, or 80-100 mM, 75-125 mM, 95-105 mM, or 97.5-102.5 mM). In some embodiments, a composition described herein comprises arginine at a concentration of about 50 mM, 55 mM, 60 mM, 65 mM, 70 mM, 75 mM, 80 mM, 85 mM, 90 mM, 95 mM, 100 mM, 105 mM, 110 mM, 115 mM, 120 mM, 125 mM, 130 mM, 135 mM, 140 mM, 145 mM, or 150 mM. In some embodiments, a composition described herein comprises arginine at a concentration of 100-150 mM. In some embodiments, a composition described herein comprises arginine at a concentration of 100 mM. In some embodiments, a composition described herein comprises arginine-HCl at a concentration of 50-150 mM (e.g., 50-125 mM, 60-120 mM, 70-110 mM, or 80-100 mM, 75-125 mM, 95-105 mM, or 97.5-102.5 mM). In some embodiments, a composition described herein comprises arginine-HCl at a concentration of about 50 mM, 55 mM, 60 mM, 65 mM, 70 mM, 75 mM, 80 mM, 85 mM, 90 mM, 95 mM, 100 mM, 105 mM, 110 mM, 115 mM, 120 mM, 125 mM, 130 mM, 135 mM, 140 mM, 145 mM, or 150 mM. In some embodiments, a composition described herein comprises arginine-HCl at a concentration of 100 mM.

In some embodiments, a composition described herein comprises a stabilizer. In some embodiments, the stabilizer is a carbohydrate. In some embodiments, the stabilizer is a sugar. In some embodiments, the stabilizer is a hexose. In some embodiments, the stabilizer is trehalose. In some embodiments, a composition described herein comprises trehalose at a concentration of 3-10% w/v (equivalent to 30-100 mg/ml). For example, a composition described herein may comprise trehalose at a concentration of 3-10% w/v, 3-9% w/v, 3-8% w/v, 3-7% w/v, 3-6% w/v, 3-5% w/v, 3-4% w/v, 3-10% w/v, 3-9% w/v, 3-8% w/v, 3-7% w/v, 3-6% w/v, 3-5% w/v, 3-4% w/v, 4-10% w/v, 4-9% w/v, 4-8% w/v, 4-7% w/v, 4-6% w/v, 4-5% w/v, 5-10% w/v, 5-9% w/v, 5-8% w/v, 5-7% w/v, 5-6% w/v, 6-10% w/v, 6-9% w/v, 6-8% w/v, 6-7% w/v, 7-10% w/v, 7-9% w/v, 7-8% w/v, 8-10% w/v, 8-9% w/v, or 9-10% w/v (equivalent to 30-100 mg/ml, 30-90 mg/ml, 30-80 mg/ml, 30-70 mg/ml, 30-60 mg/ml, 30-50 mg/ml, 30-40 mg/ml, 40-100 mg/ml, 40-90 mg/ml, 40-80 mg/ml, 40-70 mg/ml, 40-60 mg/ml, 40-50 mg/ml, 50-100 mg/ml, 50-90 mg/ml, 50-80 mg/ml, 50-70 mg/ml, 50-60 mg/ml, 60-100 mg/ml, 60-90 mg/ml, 60-80 mg/ml, 60-70 mg/ml, 70-100 mg/ml, 70-90 mg/ml, 70-80 mg/ml, 80-100 mg/ml, 80-90 mg/ml, or 90-100 mg/ml, respectively). In some embodiments, a composition described herein comprises trehalose at a concentration of about 3% w/v (equivalent to 30 mg/ml), 3.5% w/v (equivalent to 35 mg/ml), 4% w/v (equivalent to 40 mg/ml), 4.5% w/v (equivalent to 45 mg/ml), 5% w/v (equivalent to 50 mg/ml), 5.5% w/v (equivalent to 55 mg/ml), 6% w/v (equivalent to 60 mg/ml), 6.5% w/v (equivalent to 65 mg/ml), 7% w/v (equivalent to 70 mg/ml), 7.5% w/v (equivalent to 75 mg/ml), 8% w/v (equivalent to 80 mg/ml), 8.5% w/v (equivalent to 85 mg/ml), 9w/v (equivalent to 90 mg/ml), 9.5% w/v (equivalent to 95 mg/ml), or 10% w/v (equivalent to 100 mg/ml). In some embodiments, a composition described herein comprises trehalose at a concentration of about 4%-8% w/v (equivalent to 40-80 mg/ml).

In some embodiments, a composition described herein comprises trehalose at a concentration of about 4%-7% w/v (equivalent to 40-70 mg/ml). In some embodiments, a composition described herein comprises trehalose at a concentration of about 4%-6% w/v (equivalent to 40-60 mg/ml). In some embodiments, a composition described herein comprises trehalose at a concentration of about 4.5%-5.5% w/v (equivalent to 45-55 mg/ml). In some embodiments, a composition described herein comprises trehalose at a concentration of about 4% w/v, 5% w/v, 6% w/v, 7% w/v, or 8% w/v (equivalent to 40 mg/ml, 50 mg/ml, 60 mg/ml, 70 mg/ml or 80 mg/ml, respectively). In some embodiments, a composition described herein comprises trehalose at a concentration of about 5-10% w/v (equivalent to 50-100 mg/ml).

In some embodiments, a composition described herein comprises trehalose at a concentration of about 5% w/v (equivalent to 50 mg/ml).

In some embodiments, the stabilizer is sucrose. In some embodiments, a composition described herein comprises sucrose at a concentration of 3-10% w/v (equivalent to 30-100 mg/ml). For example, a composition described herein may comprise sucrose at a concentration of 3-10% w/v, 3-9% w/v, 3-8% w/v, 3-7% w/v, 3-6% w/v, 3-5% w/v, 3-4% w/v, 3-10% w/v, 3-9% w/v, 3-8% w/v, 3-7% w/v, 3-6% w/v, 3-5% w/v, 3-4% w/v, 4-10% w/v, 4-9% w/v, 4-8% w/v, 4-7% w/v, 4-6% w/v, 4-5% w/v, 5-10% w/v, 5-9% w/v, 5-8% w/v, 5-7% w/v, 5-6% w/v, 6-10% w/v, 6-9% w/v, 6-8% w/v, 5-7% w/v, 7-10% w/v, 7-9% w/v, 7-8% w/v, 8-10% w/v, 8-9% w/v, or 9-10% w/v (equivalent to 30-100 mg/ml, 30-90 mg/ml, 30-80 mg/ml, 30-70 mg/ml, 30-60 mg/ml, 30-50 mg/ml, 30-40 mg/ml, 40-100 mg/ml, 40-90 mg/ml, 40-80 mg/ml, 40-70 mg/ml, 40-60 mg/ml, 40-50 mg/ml, 50-100 mg/ml, 50-90 mg/ml, 50-80 mg/ml, 50-70 mg/ml, 50-60 mg/ml, 60-100 mg/ml, 60-90 mg/ml, 60-80 mg/ml, 60-70 mg/ml, 70-100 mg/ml, 70-90 mg/ml, 60-80 mg/ml, 80-100 mg/ml, 80-90 mg/ml, or 90-100 mg/ml, respectively). In some embodiments, a composition described herein comprises sucrose at a concentration of about 5-10% w/v (equivalent to 50-100 mg/ml).

In some embodiments, a composition described herein comprises sucrose at a concentration of about 3% w/v (equivalent to 30 mg/ml), 3.5% w/v (equivalent to 35 mg/ml), 4% w/v (equivalent to 40 mg/ml), 4.5% w/v (equivalent to 45 mg/ml), 5% w/v (equivalent to 50 mg/ml), 5.5% w/v (equivalent to 55 mg/ml), 6% w/v (equivalent to 60 mg/ml), 6.5% w/v (equivalent to 65 mg/ml), 7% w/v (equivalent to 70 mg/ml), 7.5% w/v (equivalent to 75 mg/ml), 8% w/v (equivalent to 80 mg/ml), 8.5% w/v (equivalent to 85 mg/ml), 9w/v (equivalent to 90 mg/ml), 9.5% w/v (equivalent to 95 mg/ml), or 10% w/v (equivalent to 100 mg/ml). In some embodiments, a composition described herein comprises sucrose at a concentration of about 4%-8% w/v (equivalent to 40-80 mg/ml). In some embodiments, a composition described herein comprises sucrose at a concentration of about 4%-7% w/v (equivalent to 40-70 mg/ml). In some embodiments, a composition described herein comprises sucrose at a concentration of about 4%-6% w/v (equivalent to 40-60 mg/ml). In some embodiments, a composition described herein comprises sucrose at a concentration of about 4.5%-5.5% w/v (equivalent to 45-55 mg/ml). In some embodiments, a composition described herein comprises sucrose at a concentration of about 4% w/v, 5% w/v, 6% w/v, 7% w/v, or 8% w/v (equivalent to 40 mg/ml, 50 mg/ml, 60 mg/ml, 70 mg/ml or 80 mg/ml, respectively). In some embodiments, a composition described herein comprises sucrose at a concentration of about 5% w/v (equivalent to 50 mg/ml).

In some embodiments, a composition described herein comprises a surfactant. In some embodiments, the surfactant is a polysorbate. In some embodiments, the surfactant is a polysorbate 80 (PS80). In some embodiments, a composition described herein comprises PS80 at a concentration of 0.005-0.06% w/v (equivalent to 0.05-0.6 mg/ml). In some embodiments, a composition described herein comprises PS80 at a concentration of 0.03-0.06% w/v (equivalent to 0.3-0.6 mg/ml). In some embodiments, a composition described herein comprises PS80 at a concentration of 0.04-0.06% w/v (equivalent to 0.4-0.6 mg/ml). In some embodiments, a composition described herein comprises PS80 at a concentration of 0.05-0.06% w/v (equivalent to 0.5-0.6 mg/ml). In some embodiments, a composition described herein comprises PS80 at a concentration of 0.03-0.05% w/v (equivalent to 0.3-0.5 mg/ml). In some embodiments, a composition described herein comprises PS80 at a concentration of 0.04-0.06% w/v (equivalent to 0.3-0.4 mg/ml). In some embodiments, a composition described herein comprises PS80 at a concentration of 0.03% w/v, 0.035% w/v, 0.04% w/v, 0.045% w/v, 0.05% w/v, 0.055% w/v, or 0.06% w/v. In some embodiments, a composition described herein comprises PS80 at a concentration of 0.005-0.03% w/v (equivalent to 0.05-0.3 mg/ml). For example, a composition described herein may comprise PS80 at a concentration of 0.005-0.03% w/v, 0.005-0.025% w/v, 0.005-0.02% w/v, 0.005-0.015% w/v, 0.005-0.01% w/v, 0.01-0.03% w/v, 0.01-0.025% w/v, 0.01-0.02% w/v, 0.01-0.015% w/v, 0.015-0.03% w/v, 0.015-0.025% w/v, 0.015-0.02% w/v, 0.02-0.03% w/v, 0.02-0.025% w/v, 0.02-0.03% w/v, 0.02-0.025% w/v, or 0.025-0.03% w/v (equivalent to 0.05-0.3 mg/ml, 0.05-0.25 mg/ml, 0.05-0.2 mg/ml, 0.05-0.15 mg/ml, 0.05-0.1 mg/ml, 0.1-0.3 mg/ml, 0.1-0.25 mg/ml, 0.1-0.2 mg/ml, 0.1-0.15 mg/ml, 0.15-0.3 mg/ml, 0.15-0.25 mg/ml, 0.15-0.2 mg/ml, 0.2-0.3 mg/ml, 0.2-0.25 mg/ml, or 0.25-0.3 mg/ml, respectively). In some embodiments, a composition described herein comprises PS80 at a concentration of about 0.007% w/v (equivalent to 0.07 mg/ml), 0.008% w/v (equivalent to 0.08 mg/ml), 0.009% w/v (equivalent to 0.09 mg/ml), 0.01% w/v (equivalent to 0.1 mg/ml), 0.011% w/v (equivalent to 0.11 mg/ml), 0.012% w/v (equivalent to 0.12 mg/ml), 0.013% w/v (equivalent to 0.13 mg/ml), 0.014% w/v (equivalent to 0.14 mg/ml), 0.015% w/v (equivalent to 0.15 mg/ml), 0.016% w/v (equivalent to 0.16 mg/ml), 0.017% w/v (equivalent to 0.17 mg/ml), 0.018% w/v (equivalent to 0.18 mg/ml), 0.019w/v (equivalent to 0.19 mg/ml), or 0.02% w/v (equivalent to 0.2 mg/ml). In some embodiments, a composition described herein comprises PS80 at a concentration of about 0.01%-0.03% w/v (equivalent to 0.1-0.3 mg/ml). In some embodiments, a composition described herein comprises PS80 at a concentration of about 0.015%-0.025% w/v (equivalent to 0.15-0.25 mg/ml). In some embodiments, a composition described herein comprises PS80 at a concentration of about 0.02% w/v (equivalent to 0.2 mg/ml).

In some embodiments, a composition described herein comprises a CD38-binding fusion protein (e.g., a CD38-binding fusion protein as provided in Table 1) at a concentration of 8.5-11.5 mg/ml (e.g., 10 mg/ml), histidine (e.g., composed of histidine and histidine-HCl) at a concentration of 15-60 mM (e.g., 15 mM, 20 mM, 30 mM, 40 mM, or 50 mM), arginine-HCl at a concentration of 80-120 mM (e.g., 100 mM), sucrose at a concentration of 3-8% w/v (e.g., 5% w/v), and PS80 at a concentration of 0.01-0.03% w/v (e.g., 0.02% w/v), and wherein the composition is at a pH of 5.5-7.5 (e.g., 5.5, 6, 6.5, or 6.6). In some embodiments, the CD38-binding fusion protein comprises a heavy chain comprising the amino acid sequence of SEQ ID NO: 13 and a light chain comprising the amino acid sequence of SEQ ID NO: 10.

In some embodiments, a composition described herein comprises a CD38-binding fusion protein (e.g., a CD38-binding fusion protein as provided in Table 1) at a concentration of 30-80 mg/ml, histidine (e.g., composed of histidine and histidine-HCl) at a concentration of 50-75 mM (e.g., 50 mM), arginine-HCl at a concentration of 75-150 mM (e.g., 100 mM), sucrose at a concentration of 3-10% w/v (e.g., 5% w/v), and PS80 at a concentration of 0.01-0.06% w/v (e.g., 0.02% w/v), and wherein the composition is at a pH of 6.0-7.0 (e.g., 6.5-6.7). In some embodiments, the CD38-binding fusion protein comprises a heavy chain comprising the amino acid sequence of SEQ ID NO: 13 and a light chain comprising the amino acid sequence of SEQ ID NO: 10.

In some embodiments, a composition described herein comprises a CD38-binding fusion protein (e.g., a CD38-binding fusion protein as provided in Table 1) at a concentration of 40-80 mg/ml, histidine (e.g., composed of histidine and histidine-HCl) at a concentration of 50-75 mM (e.g., 50 mM), arginine-HCl at a concentration of 75-150 mM (e.g., 100 mM), sucrose at a concentration of 3-10% w/v (e.g., 5% w/v), and PS80 at a concentration of 0.01-0.06% w/v (e.g., 0.02% w/v), and wherein the composition is at a pH of 6.0-7.0 (e.g., 6.5-6.7). In some embodiments, the CD38-binding fusion protein comprises a heavy chain comprising the amino acid sequence of SEQ ID NO: 13 and a light chain comprising the amino acid sequence of SEQ ID NO: 10.

In some embodiments, a composition described herein comprises a CD38-binding fusion protein (e.g., a CD38-binding fusion protein as provided in Table 1) at a concentration of 30 mg/ml, histidine (e.g., composed of histidine and histidine-HCl) at a concentration of 50-75 mM (e.g., 50 mM), arginine-HCl at a concentration of 75-150 mM (e.g., 100 mM), sucrose at a concentration of 3-10% w/v (e.g., 5% w/v), and PS80 at a concentration of 0.01-0.06% w/v (e.g., 0.02% w/v), and wherein the composition is at a pH of 6.0-7.0 (e.g., 6.5-6.7). In some embodiments, the CD38-binding fusion protein comprises a heavy chain comprising the amino acid sequence of SEQ ID NO: 13 and a light chain comprising the amino acid sequence of SEQ ID NO: 10.

In some embodiments, a composition described herein comprises a CD38-binding fusion protein (e.g., a CD38-binding fusion protein as provided in Table 1) at a concentration of 40 mg/ml, histidine (e.g., composed of histidine and histidine-HCl) at a concentration of 50-75 mM (e.g., 50 mM), arginine-HCl at a concentration of 75-150 mM (e.g., 100 mM), sucrose at a concentration of 3-10% w/v (e.g., 5% w/v), and PS80 at a concentration of 0.01-0.06% w/v (e.g., 0.02% w/v), and wherein the composition is at a pH of 6.0-7.0 (e.g., 6.5-6.7). In some embodiments, the CD38-binding fusion protein comprises a heavy chain comprising the amino acid sequence of SEQ ID NO: 13 and a light chain comprising the amino acid sequence of SEQ ID NO: 10.

In some embodiments, a composition described herein comprises a CD38-binding fusion protein (e.g., a CD38-binding fusion protein as provided in Table 1) at a concentration of 60 mg/ml, histidine (e.g., composed of histidine and histidine-HCl) at a concentration of 50-75 mM (e.g., 50 mM), arginine-HCl at a concentration of 75-150 mM (e.g., 100 mM), sucrose at a concentration of 3-10% w/v (e.g., 5% w/v), and PS80 at a concentration of 0.01-0.06% w/v (e.g., 0.02% w/v), and wherein the composition is at a pH of 6.0-7.0 (e.g., 6.5-6.7). In some embodiments, the CD38-binding fusion protein comprises a heavy chain comprising the amino acid sequence of SEQ ID NO: 13 and a light chain comprising the amino acid sequence of SEQ ID NO: 10.

In some embodiments, a composition described herein comprises a CD38-binding fusion protein (e.g., a CD38-binding fusion protein as provided in Table 1) at a concentration of 80 mg/ml, histidine (e.g., composed of histidine and histidine-HCl) at a concentration of 50-75 mM (e.g., 50 mM), arginine-HCl at a concentration of 75-150 mM (e.g., 100 mM), sucrose at a concentration of 3-10% w/v (e.g., 5% w/v), and PS80 at a concentration of 0.01-0.06% w/v (e.g., 0.02% w/v), and wherein the composition is at a pH of 6.0-7.0 (e.g., 6.5-6.7). In some embodiments, the CD38-binding fusion protein comprises a heavy chain comprising the amino acid sequence of SEQ ID NO: 13 and a light chain comprising the amino acid sequence of SEQ ID NO: 10.

In some embodiments, a composition described herein comprises a CD38-binding fusion protein (e.g., a CD38-binding fusion protein as provided in Table 1) at a concentration of 100 mg/ml, histidine (e.g., composed of histidine and histidine-HCl) at a concentration of 50-75 mM (e.g., 50 mM), arginine-HCl at a concentration of 75-150 mM (e.g., 100 mM), sucrose at a concentration of 3-10% w/v (e.g., 5% w/v), and PS80 at a concentration of 0.01-0.06% w/v (e.g., 0.02% w/v), and wherein the composition is at a pH of 6.0-7.0 (e.g., 6.5-6.7). In some embodiments, the CD38-binding fusion protein comprises a heavy chain comprising the amino acid sequence of SEQ ID NO: 13 and a light chain comprising the amino acid sequence of SEQ ID NO: 10.

In some embodiments, a composition described herein comprises a CD38-binding fusion protein (e.g., a CD38-binding fusion protein as provided in Table 1) at a concentration of 10 mg/ml, histidine (e.g., composed of histidine and histidine-HCl) at a concentration of 50 mM, arginine-HCl at a concentration of 100 mM, sucrose at a concentration of 5% w/v, and PS80 at a concentration of 0.02% w/v, and wherein the composition is at a pH of 6.6. In some embodiments, the CD38-binding fusion protein comprises a heavy chain comprising the amino acid sequence of SEQ ID NO: 13 and a light chain comprising the amino acid sequence of SEQ ID NO: 10.

In some embodiments, a composition described herein comprises a CD38-binding fusion protein (e.g., a CD38-binding fusion protein as provided in Table 1) at a concentration of 10 mg/ml, histidine (e.g., composed of histidine and histidine-HCl) at a concentration of 15 mM, arginine-HCl at a concentration of 100 mM, sucrose at a concentration of 5% w/v, and PS80 at a concentration of 0.02% w/v, and wherein the composition is at a pH of 6. In some embodiments, the CD38-binding fusion protein comprises a heavy chain comprising the amino acid sequence of SEQ ID NO: 13 and a light chain comprising the amino acid sequence of SEQ ID NO: 10.

In some embodiments, a composition described herein is an aqueous solution. In some embodiments, the present disclosure provides a lyophilized form of a composition described herein. In some embodiments, the lyophilized form can be stored, and reconstituted to an aqueous solution, prior to use (e.g., administration to a subject).

In some embodiments, a composition described herein (e.g., in a form of aqueous solution or in lyophilized form) is stored in dosage unit form. In some embodiments, a lyophilized form of a composition described herein is stored for at least 2 months, at least 4 months, at least 6 months, at least 1 year, at least 2 years, or at least 3 years. In some embodiments, a composition described herein (e.g., in a form of aqueous solution or in lyophilized form) is stored frozen. In some embodiments, a composition described herein (e.g., in a form of aqueous solution or in lyophilized form) is stored at room temperature (e.g., 25-40° C.). In some embodiments, a composition described herein (e.g., in a form of aqueous solution or in lyophilized form) is stored at a temperature under 40° C.

In some embodiments, at least 80% (e.g., at least 80%, at least 85%, at least 90%, at least 95%, or at least 99%) of the CD38-binding fusion protein in a composition described herein remains intact (e.g., not degraded) after being in storage (e.g., at −30-40° C.) for at least 3 months (e.g., at least 3 months, at least 6 months, or at least 1 year).

In some embodiments, less than 5% (e.g., less than 5%, less than 4%, less than 3%, or less than 2%, less than 1%, less than 0.5%, or less than 0.25%) of the CD38-binding fusion protein in a composition described herein forms dimers after being in storage (e.g., at −30-40° C.) for at least 3 months (e.g., at least 3 months, at least 6 months, or at least 1 year).

In some embodiments, less than 5% (e.g., less than 5%, less than 4%, less than 3%, or less than 2%, less than 1%, less than 0.5%, or less than 0.25%) of the CD38-binding fusion protein in a composition described herein aggregates after being in storage (e.g., at −30-40° C.) for at least 3 months (e.g., at least 3 months, at least 6 months, or at least 1 year).

In some embodiments, the pH of a composition described herein varies by less than 0.5 (e.g., by less than 0.5, by less than 0.4, or by less than 0.3) after being in storage (e.g., at −30-40° C.) for at least 3 months (e.g., at least 3 months, at least 6 months, or at least 1 year).

In some embodiments, a composition described herein is in a lyophilized form for storage, and the moisture content of the lyophilized composition is less than 3% (e.g., less than 3%, or less than 2%) after being in storage (e.g., at −30-40° C.) for at least 3 months (e.g., at least 3 months, at least 6 months, or at least 1 year).

In some embodiments, a composition described herein retains at least 60% (e.g., at least 60%, at least 65%, at least 70%, at least 75%, at least 80%, at least 85%, at least 90%, at least 95%, or at least 99%) of activity (e.g., as indicated by a cell-based potency assay) after being in storage (e.g., at −30 to −15° C.) for at least 3 months (e.g., at least 3 months, at least 6 months, or at least 1 year), relative to a composition before storage.

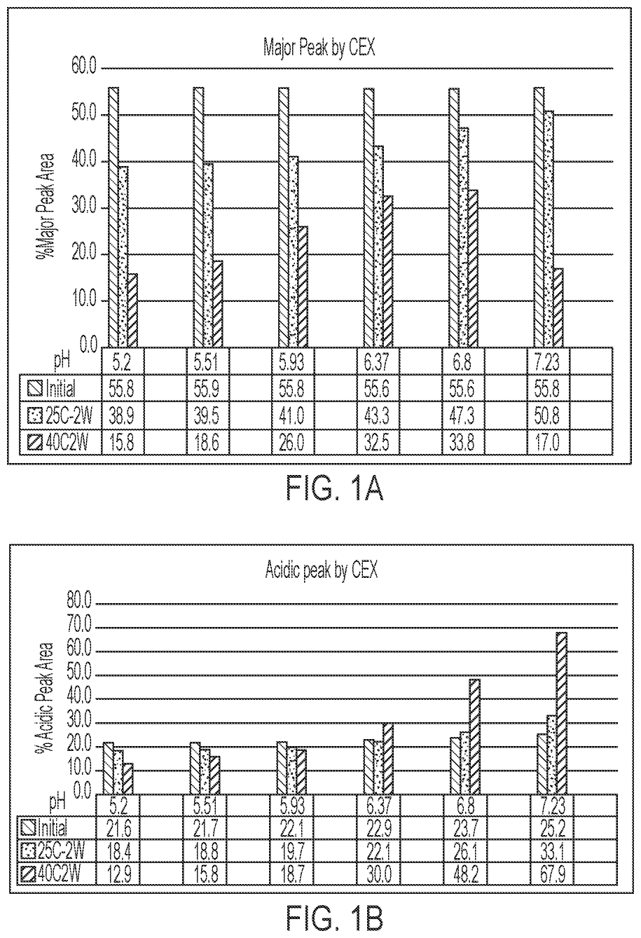

In some embodiments, a composition described herein comprises a relative percentage of a main peak of a CD38-binding protein (e.g., as determined using cation exchange chromatography) that changes no more than 10%-20% (e.g., decreases by no more than 15%) after 2 weeks of storage (e.g., lyophilized storage) at 25° C. In some embodiments, a composition described herein comprises a relative percentage of a main peak of a CD38-binding protein (e.g., as determined using cation exchange chromatography) that changes no more than 20-30% (e.g., decreases by no more than 25%) after 2 weeks of storage (e.g., lyophilized storage) at 40° C. A composition as described herein in reference to FIG. 1 or a composition described herein having a pH of 5.8-7.0 (e.g., a pH of 6.4-6.8) may have these properties.

In some embodiments, a composition described herein comprises a relative percentage of an acidic peak of a CD38-binding protein (e.g., as determined using cation exchange chromatography) that changes no more than 2.5-7.5% (e.g., by no more than 5%) after 2 weeks of storage (e.g., lyophilized storage) at 25° C. In some embodiments, a composition described herein comprises a relative percentage of an acidic peak of a CD38-binding protein (e.g., as determined using cation exchange chromatography) that changes no more than 10-30% (e.g., by no more than 10% or 30%) after 2 weeks of storage (e.g., lyophilized storage) at 40° C. A composition as described herein in reference to FIG. 1 or a composition described herein having a pH of 5.8-7.0 (e.g., a pH of 6.4-6.8) may have these properties.

In some embodiments, a composition described herein comprises a relative percentage of a basic peak of a CD38-binding protein (e.g., as determined using cation exchange chromatography) that changes by no more than 2-20% (e.g., by no more than 10% or 15%) after 2 weeks of storage (e.g., lyophilized storage) at 25° C. In some embodiments, a composition described herein comprises a relative percentage of a basic peak of a CD38-binding protein (e.g., as determined using cation exchange chromatography) that changes no more than 5-30% (e.g., by no more than 5% or 30%) after 2 weeks of storage (e.g., lyophilized storage) at 40° C. A composition as described herein in reference to FIG. 1 or a composition described herein having a pH of 6.0-7.3 (e.g., a pH of 6.4-6.8) may have these properties.

In some embodiments, a composition described herein comprises a relative percentage of aggregate (e.g., as determined by size exclusion chromatography) that changes no more than 0.04-0.15% (e.g., increases by no more than 0.1%) after 2 weeks of storage (e.g., lyophilized storage) at 25° C. In some embodiments, a composition described herein comprises a relative percentage of aggregate (e.g., as determined by size exclusion chromatography) that changes no more than 0.3-2% (e.g., increases by no more than 1.5%) after 2 weeks of storage (e.g., lyophilized storage) at 40° C. A composition as described herein in reference to FIG. 1 or a composition described herein having a pH of 5.2-7.0 (e.g., a pH of 6.4-6.8) may have these properties.

In some embodiments, a composition described herein comprises a relative percentage of low molecular weight molecules (LMW) (e.g., as determined by size exclusion chromatography) that changes by no more than 0.03-0.1% (e.g., by no more than 0.06%) after 2 weeks of storage (e.g., lyophilized storage) at 25° C. In some embodiments, a composition described herein comprises a relative percentage of low molecular weight molecules (LMW) (e.g., as determined by size exclusion chromatography) that changes by no more than 0.02-0.1% (e.g., by no more than 0.07%) after 2 weeks of storage (e.g., lyophilized storage) at 40° C. A composition as described herein in reference to FIG. 1 or a composition described herein having a pH of 5.4-7.3 (e.g., a pH of 6.4-6.8) may have these properties.

In some embodiments, a composition described herein comprises a relative percentage of CD38-binding protein monomer (e.g., as determined by size exclusion chromatography) that changes by no more than 0.01-0.2% (e.g., by no more than 0.07%) after 2 weeks of storage (e.g., lyophilized storage) at 25° C. In some embodiments, a composition described herein comprises a relative percentage of CD38-binding protein monomer (e.g., when determined by size exclusion chromatography) that changes by no more than 0.2-1.5% (e.g., by no more than 0.6% or 1.3%) after 2 weeks of storage (e.g., lyophilized storage) at 40° C. A composition as described herein in reference to FIG. 1 or a composition described herein having a pH of 5.0-7.0 (e.g., a pH of 6.4-6.8) may have these properties.

In some embodiments, a composition described herein comprises a relative percentage of CD38-binding protein monomer (e.g., as determined by size exclusion chromatography) that changes by no more than 0.2-0.5% (e.g., by no more than 0.4%) after 4 months of storage (e.g., lyophilized storage) at 2-8° C. A composition as described herein in reference to Table 2 or a composition described herein having an arginine concentration of 25-150 mM arginine (e.g., 100 mM arginine) and a pH of 6.0-7.0 (e.g., a pH of 6.4-6.8) may have these properties.

In some embodiments, a composition described herein comprises a relative percentage of CD38-binding protein monomer (e.g., as determined by size exclusion chromatography) that changes by no more than 0.2-0.5% (e.g., by no more than 0.4%) after 1 month of storage (e.g., lyophilized storage) at 2-8° C. A composition as described herein in reference to Table 2 or a composition described herein having an arginine concentration of 25-150 mM arginine (e.g., 25-100 mM arginine) and a pH of 6.0-7.0 (e.g., a pH of 6.4-6.8) may have these properties.

In some embodiments, a composition described herein comprises a relative percentage of CD38-binding protein monomer (e.g., as determined by size exclusion chromatography) that changes by no more than 2-6% (e.g., by no more than 5%) after 4 months of storage (e.g., lyophilized storage) at 40° C. A composition as described herein in reference to Table 2 or a composition described herein having an arginine concentration of 100-150 mM arginine (e.g., 100 mM arginine) and a pH of 6.0-7.0 (e.g., a pH of 6.4-6.8) may have these properties.

In some aspects, the present disclosure relates to methods of treating cancer in a subject by administering an effective amount of a composition described herein. In some embodiments, an effective amount of the composition is administered to a patient with cancer. In some embodiments, an effective amount is the amount required to treat a cancer in a patient. Treating may include, for example, inhibiting or reducing proliferation of CD38-positive cells in the cancer and/or inducing apoptosis of CD38-positive cells in the cancer.

The terms “subject” and “patient” are used interchangeably and include any mammals, including companion and farm mammals, as well as rodents, including mice, rabbits, and rats, and other rodents. Non-human primates, such as Cynomolgus monkeys, are more preferred, and human beings are highly preferred.

The term “about” means within 5%, e.g., within 5%, 4%, 3%, 2%, or 1% of a given value or range. The terms “treatment”, “treating”, “treat”, and the like, refer to obtaining a desired pharmacologic and/or physiologic effect. The effect may be prophylactic in terms of completely or partially preventing a disease or symptom thereof or reducing the likelihood of a disease or symptom thereof and/or may be therapeutic in terms of a partial or complete cure for a disease and/or adverse effect attributable to the disease. “Treatment”, as used herein, covers any treatment of a disease in a mammal, particularly In a human, and includes: (a) preventing the disease from occurring in a subject which may be predisposed to the disease but has not yet been diagnosed as having it; (b) inhibiting the disease, i.e., arresting its development or progression; and (c) relieving the disease, i.e., causing regression of the disease and/or relieving one or more disease symptoms. “Treatment” is also meant to encompass delivery of an agent in order to provide for a pharmacologic effect, even in the absence of a disease or condition.

An “effective amount” or “therapeutically effective amount” of a composition includes that amount of the composition which is sufficient to provide a beneficial effect to the subject to which the composition is administered. An “effective amount” of a delivery vehicle includes that amount sufficient to effectively bind or deliver a composition.

Any composition described or exemplified herein may be used for treatment of the disorders described herein. Tumors that may be treated include, but are not limited to AIDS related cancers, acoustic neuroma, acute lymphocytic leukemia, acute myeloid leukemia, adenocystic carcinoma, adrenocortical cancer, agnogenic myeloid metaplasia, alopecia, alveolar soft-part sarcoma, anal cancer, angiosarcoma, aplastic anemia, astrocytoma, ataxia-telangiectasia, basal cell carcinoma (skin), bladder cancer, bone cancers, bowel cancer, brain stem glioma, brain and CNS tumors, breast cancer, CNS tumors, carcinoid tumors, cervical cancer, childhood brain tumors, childhood cancer, childhood leukemia, childhood soft tissue sarcoma, chondrosarcoma, choriocarcinoma, chronic lymphocytic leukemia, chronic myeloid leukemia, colorectal cancers, cutaneous T-Cell lymphoma, dermatofibrosarcoma-protuberans, desmoplastic-small-round-cell-tumor, ductal carcinoma, endocrine cancers, endometrial cancer, ependymoma, esophageal cancer, Ewing's sarcoma, extra-hepatic bile duct cancer, eye cancer, eye: melanoma, retinoblastoma, fallopian tube cancer, fanconi anemia, fibrosarcoma, gall bladder cancer, gastric cancer, gastrointestinal cancers, gastrointestinal-carcinoid-tumor, genitourinary cancers, germ cell tumors, gestational-trophoblastic-disease, glioma, gynecological cancers, hematological malignancies, hairy cell leukemia, head and neck cancer, hepatocellular cancer, hereditary breast cancer, histiocytosis, Hodgkin's disease, human papillomavirus, hydatidiform mole, hypercalcemia, hypopharynx cancer, intraocular melanoma, islet cell cancer, Kaposi's sarcoma, kidney cancer, Langerhan's-cell-histiocytosis, laryngeal cancer, leiomyosarcoma, leukemia, Li-Fraumeni syndrome, lip cancer, liposarcoma, liver cancer, lung cancer, lymphedema, lymphoma, Hodgkin's lymphoma, non-Hodgkin's lymphoma, male breast cancer, malignant-rhabdoid-tumor-of-kidney, medulloblastoma, melanoma, merkel cell cancer, mesothelioma, metastatic cancer, mouth cancer, multiple endocrine neoplasia, mycosis fungoides, myelodysplastic syndromes, multiple myeloma, myeloproliferative disorders, nasal cancer, nasopharyngeal cancer, nephroblastoma, neuroblastoma, neurofibromatosis, nijmegen breakage syndrome, non-melanoma skin cancer, non-small-cell-lung-cancer-(NSCLC), ocular cancers, esophageal cancer, oral cavity cancer, oropharynx cancer, osteosarcoma, ostomy ovarian cancer, pancreas cancer, paranasal cancer, parathyroid cancer, parotid gland cancer, penile cancer, peripheral-neuroectodermal-tumors, pituitary cancer, polycythemia vera, prostate cancer, rare-cancers-and-associated-disorders, renal cell carcinoma, retinoblastoma, rhabdomyosarcoma, Rothmund-Thomson syndrome, salivary gland cancer, sarcoma, schwannoma, Sezary syndrome, skin cancer, small cell lung cancer (SCLC), small intestine cancer, soft tissue sarcoma, spinal cord tumors, squamous-cell-carcinoma-(skin), stomach cancer, synovial sarcoma, testicular cancer, thymus cancer, thyroid cancer, transitional-cell-cancer-(bladder), transitional-cell-cancer-(renal-pelvis-/-ureter), trophoblastic cancer, urethral cancer, urinary system cancer, uroplakins, uterine sarcoma, uterus cancer, vaginal cancer, vulva cancer, Waldenstrom's-macroglobulinemia and Wilms' tumor. In an embodiment the tumor is selected from a group of multiple myeloma or non-Hodgkin's lymphoma.

In some embodiments, the methods are used for treatment of multiple myeloma, leukemia, or lymphoma in a subject in need thereof. Such methods may further comprise treating the subject with a retinoid, such as all-trans retinoic acid. In some preferred aspects in which the cell surface associated antigen is CD38, the tumor or cancer may be selected from multiple myeloma, non-Hodgkin's lymphoma, chronic myelogenous leukemia, chronic lymphocytic leukemia or acute myelogenous leukemia.

A composition described herein may be combined with other drugs and/or used in addition to other cancer treatment regimens or modalities such as radiation therapy or surgery. When CD38-binding fusion protein are used in combination with known therapeutic agents the combination may be administered either in sequence (either continuously or broken up by periods of no treatment) or concurrently or as a mixture. In the case of cancer, there are numerous known anticancer agents that may be used in this context. Treatment in combination is also contemplated to encompass the treatment with either the CD38-binding fusion protein followed by a known treatment, or treatment with a known agent followed by treatment with the CD38-binding fusion protein, for example, as maintenance therapy. For example, in the treatment of cancer it is contemplated that the CD38-binding fusion protein may be administered in combination with an alkylating agent (such as mechlorethamine, cyclophosphamide, chlorambucil, ifosfamidecysplatin, or platinum-containing alkylating-like agents such as cisplatin, carboplatin and oxaliplatin), an antimetabolite (such as a purine or pyrimidine analogue or an antifolate agent, such as azathioprine and mercaptopurine), an anthracycline (such as Daunorubicin, Doxorubicin, Epirubicinldarubicin, Valrubicin, Mitoxantrone, or anthracycline analog), a plant alkaloid (such as a vinca alkaloid or a taxane, such as Vincristine, Vinblastine, Vinorelbine, Vindesine, paclitaxel or Dosetaxel), a topoisomerase inhibitor (such as a type I or type II topoisomerase inhibitor), a Podophyllotoxin (such as etoposide or teniposide), or a tyrosine kinase inhibitor (such as imatinibmesylate, Nilotinib, or Dasatinib).

In the case of the treatment of multiple myeloma, a composition described herein may be administered in combination with other suitable therapies, such as treatment of the subject with the administration of steroids such as dexamethasone, proteasome inhibitors (such as bortezomib or carfilzomib), immunomodulatory drugs (such as thalidomide, lenalidomide or pomalidomide), or induction chemotherapy followed by autologous hematopoietic stem cell transplantation, with or without other chemotherapeutic agents such as Melphalan hydrochloride or the chemotherapeutic agents listed above.

In the case of the treatment of Hodgkin's lymphoma, a composition described herein may be administered in combination with current therapeutic approaches, such as ABVD (Adriamycin (doxorubicin), bleomycin, vinblastine, and dacarbazine), or Stanford V (doxorubicin, bleomycin, vinblastine, vincristine, mechlorethamine, etoposide, prednisone), or BEACOPP (doxorubicin, bleomycin, vincristine, cyclophosphamide, procarbazine, etoposide, prednisone).

In the case of non-Hodgkin's lymphoma or other lymphomas, a composition described herein may be administered in combination current therapeutic approaches. Examples of drugs approved for non-Hodgkin lymphoma include Abitrexate (Methotrexate), Adriamycin PFS (Doxorubicin Hydrochloride), Adriamycin RDF (Doxorubicin Hydrochloride), Ambochlorin (Chlorambucil), Amboclorin (Chlorambucil), Arranon (Nelarabine), Bendamustine Hydrochloride, Bexxar (Tositumomab and Iodine I¹³¹ Tositumomab), Blenoxane (Bleomycin), Bleomycin, Bortezomib, Chlorambucil, Clafen (Cyclophosphamide), Cyclophosphamide, Cytoxan (Cyclophosphamide), DenileukinDiftitox, DepoCyt (Liposomal Cytarabine), Doxorubicin Hydrochloride, DTIC-Dome (Dacarbazine), Folex (Methotrexate), Folex PFS (Methotrexate), Folotyn (Pralatrexate), Ibritumomab Tiuxetan, Istodax (Romidepsin), Leukeran (Chlorambucil), Linfolizin (Chlorambucil), Liposomal Cytarabine, Matulane (Procarbazine Hydrochloride), Methotrexate, Methotrexate LPF (Methotrexate), Mexate (Methotrexate), Mexate-AQ (Methotrexate), Mozobil (Plerixafor), Nelarabine, Neosar (Cyclophosphamide), Ontak (DenileukinDiftitox), Plerixafor, Pralatrexate, Rituxan (Rituximab), Rituximab, Romidepsin, Tositumomab and Iodine I 131 Tositumomab, Treanda (Bendamustine Hydrochloride), Velban (Vinblastine Sulfate), Velcade (Bortezomib), and Velsar (Vinblastine Sulfate), Vinblastine Sulfate, Vincasar PFS (Vincristine Sulfate), Vincristine Sulfate, Vorinostat, Zevalin (IbritumomabTiuxetan), Zolinza (Vorinostat). Examples of drug combinations used in treating non-Hodgkin lymphoma include CHOP (C=Cyclophosphamide, H=Doxorubicin Hydrochloride (Hydroxydaunomycin), O=Vincristine Sulfate (Oncovin), P=Prednisone); COPP (C=Cyclophosphamide, O=Vincristine Sulfate (Oncovin), P=Procarbazine Hydrochloride, P=Prednisone); CVP (C-Cyclophosphamide, V=Vincristine Sulfate, P-Prednisone); EPOCH (E=Etoposide, P=Prednisone, O=Vincristine Sulfate (Oncovin), C=Cyclophosphamide, H-Doxorubicin Hydrochloride (Hydroxydaunomycin)); ICE (I=Ifosfamide, C=Carboplatin, E=Etoposide) and R-CHOP (R=Rituximab, C=Cyclophosphamide, H=Doxorubicin Hydrochloride (Hydroxydaunomycin), O=Vincristine Sulfate (Oncovin), P=Prednisone.

In some embodiments, a method of treating cancer described herein further comprises administering to the subject lenalidomide or pomalidomide, as described in U.S. Pat. No. 9,636,334, which is incorporated by reference in its entirety.

The tumor may be B-cell lymphoma, multiple myeloma, early stage multiple myeloma, pre-multiple myeloma, Waldenstrom's macroglobulinemia, non-Hodgkin's lymphoma, chronic myelogenous leukemia, chronic lymphocytic leukemia, or acute lymphocytic leukemia. In some embodiments, a composition comprising an CD38-binding fusion protein described herein is administered separately from lenalidomide or pomalidomide (e.g., lenalidomide or pomalidomide may be separately formulated in a different composition). In some embodiments, a composition comprising an CD38-binding fusion protein described herein is administered to a subject already receiving lenalidomide or pomalidomide treatment. In some embodiments, lenalidomide or pomalidomide is administered to a subject already receiving a treatment with a composition comprising an CD38-binding fusion protein described herein.

In some embodiments, administering a composition comprising an CD38-binding fusion protein described herein, and lenalidomide or pomalidomide result in synergistic effects for treating a tumor. In some embodiments, the synergistic effects may include, without limitation, lower effective dose for lenalidomide or pomalidomide, and/or the composition comprising an CD38-binding fusion protein described herein, enhanced efficacy in inhibiting tumor growth, and/or improved survival of the subjects.

In some embodiments, a method of treating cancer described herein further comprises administering to the subject a CD47 antagonist, e.g., an agent that reduces CD47 signaling, as described in PCT Patent Publication No. WO2018014067A1, which is incorporated by reference in its entirety. In some embodiments, the CD47 antagonist is an anti-CD47 antibody. In some embodiments, a method of treating cancer described herein further comprises administering to the subject a CD47 antagonist which inhibits the interaction of CD47 with the SIRPα receptor.

### EXAMPLES

### Example 1—CD38-Binding Fusion Protein Compositions

CD38 is transmembrane glycoprotein expressed at high levels in numerous types of immune cell cancers. A CD38-binding fusion protein was previously developed for treatment of CD38 expressing immune cell cancers. The CD38-binding fusion protein comprises an anti-CD38 antibody and an attenuated interferon alpha-2b protein. The CD38 antibody delivers the attenuated interferon alpha-2b protein to CD38 expressing cancers (e.g., multiple myeloma), which in turn triggers apoptosis and/or reduced proliferation of CD38 expressing cells and hence limits disease progression.

Determining protein drug formulations is a challenging part of protein drug development. A protein drug formulation protects and maintains the protein drug stability, so the protein drug can be safely administered to a patient. Protein drug formulations can be difficult to predict for a given protein drug because small changes in the protein drug's environment (e.g., changes in temperature, pH, or salinity) can cause the protein drug to denature, degrade, and/or aggregate, which reduces therapeutic efficacy and may pose safety concerns for patients. Further, different protein drugs have varying different properties (e.g., different sizes, shapes, thermodynamic properties and activities), which makes it difficult to predict whether a given formulation will be sufficient to maintain the stability and activity of a protein drug based on formulations of other protein drugs.

Compositions were developed for the storage and clinical use of CD38-binding fusion proteins. The compositions tested comprised a CD38-binding fusion protein containing sequences as provided in Table 1, histidine buffer, an arginine tonicity agent, a sucrose stabilizer, and a PS80 surfactant. Experiments were performed with varying amounts of these components at multiple temperatures, multiple pHs, and over different storage conditions and times. These compositions were lyophilized for storage and reconstituted for testing. The goal of these experiments was to identify concentration ranges of each component of the composition where the quality attributes of the composition (e.g., aggregation, subvisible particle formation, turbidity, etc.) were clinically suitable and have little to no change. Quality attributes quantified include CD38-binding fusion protein concentration, potency, aggregation, ion relative abundance and dimerization in the composition over time, and subvisible particle formation, turbidity, and pH stability of the composition over time.

Quality attributes were quantified as follows: CD38-binding fusion protein concentration was determined using UV spectroscopy by SoloVPE or absorbance at 280 nm. CD38-binding fusion protein monomer, aggregation, and dimerization were quantified using size exclusion chromatography (SEC). CD38-binding fusion protein charge profile (Main peak, acidic peak and basic peak) were quantified using cation exchange chromatography (CEX). Subvisible particle formation was measured using micro-flow imaging (MFI) particle analysis. MFI quantifies both the relative number of particles and their relative sizes. CD38-binding fusion protein aggregation and degradation was measured by turbidity. CD38-binding fusion protein potency was quantified in a cell-based potency assay. The cell-based potency assay measures multiple myeloma in vitro cell growth arrest. Specifically, the multiple myeloma cell line H929 was subcloned to produce a sub clonal H929 cell line, which is the cell line used in the assay. Sub clonal H929 cell line is identical to the parental cell line when evaluated by short tandem repeat analysis. The anti-CD38 portion of the therapeutic binds to the sub clonal H929 cells expressing the CD38 antigen on its cell surface. The attenuated interferon a portion of the therapeutic is then in proximity to and activates interferon receptors. Both the binding of anti-CD38 and activation of interferon receptors is required for a robust cell growth arrest response, leading to a reduction in intracellular ATP concentration. Cellular ATP concentration is detected using luminescent output. The assay is performed using a series of concentrations of the CD38-binding fusion protein of Table 1 test samples (TS) and reference standards (RS). The relative potency (% RP) is calculated from the ratio of EC50 values of RS and TS. The reportable value (RV) is the mean % RP.

Experiments were used to determine the pH stability of the CD38-binding fusion protein in a solutions comprising 1 mg/ml CD38-binding fusion protein containing sequences as provided in Table 1, 2.34 mM citrate, 5 mM phosphate-Na buffer, and 50 mM NaCl. Six different pH levels were tested: pH 5.2, 5.51, 5.93, 6.37, 6.8 and 7.23. Measurements were taken upon making the composition, after 2 weeks at 25° C., and after 2 weeks at 40° C. CD38-binding fusion protein pH stability was first determined by measuring the relative percentage of the main, acidic and basic peaks of CD38-fusion binding proteins in solutions of different pH (FIG. 1A-1C). The goal was to identify pH levels where the change in the relative percentages of the main, acidic and basic peaks was relatively small. Results show that pH 6.37 and pH 6.8 have similar fraction of the major, acidic and basic peaks compared to the other pHs tested. Aggregation of the CD38-binding fusion protein was also measured across pH levels (FIG. 1D).

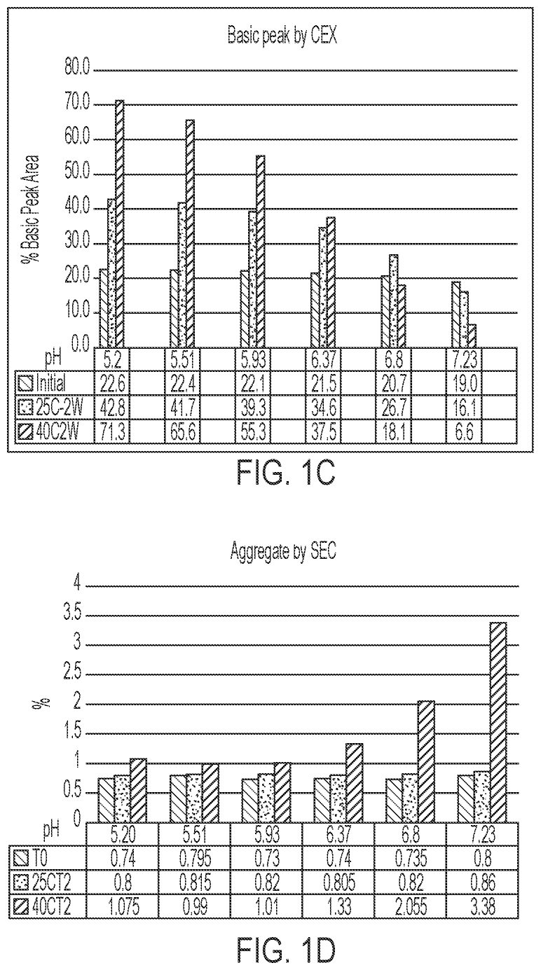

Results show that the amount of aggregation remains approximately the same at the initial and 2 week 25° C. timepoint regardless of pH. However, at the 40° C. 2 week time point increased aggregation was observed starting at pH 6.37. CD38-fusion protein degradation was also measured at different pH levels (FIG. 1E). Results show that LMW (as measured by size-exclusion chromatography) was relatively stable across all pHs, times and temperatures tested. Lastly, the concentration of the CD38-fusion protein was measured across the different pH levels (FIG. 1F). Results show that the amount of CD38-binding fusion protein in the composition is relatively stable across all pH's tested with a slight decline in the 40° C. 2 week condition at pH 6.8 and 7.23. Stability of the CD38-binding fusion protein at pH 5.1, 5.5, 6.1, 6.5, 7.1, 7.5 and 10 was also determined using calorimetry (FIG. 2 shows data for pH 5.1, 6.5 and 10). The following CD38-binding fusion protein melting temperatures were measured: pH 5.1 (65.79° C.), pH 5.5 (66.05° C.), pH 6.1 (66.27° C.), pH 6.5 (66.4° C.), pH 7.1 (66.37° C.), pH 7.5 (66.17° C.) and pH 10 (66.15° C.). Overall these results suggest that increasing pH generally increases CD38-binding fusion protein stability between pH 5.1 and pH 6.5. The results suggest that CD38-binding fusion protein is stable between pH 6.5 and 7.1 and slightly decreases after pH 7.5.

Experiments were also performed to determine the amount of arginine needed to decrease CD38-binding fusion protein precipitation. The initial composition comprised 10 mM histidine buffer, 9.3%, 7.5%, or 2.10% trehalose dihydrate, 0.02% PS80, and 0 mM, 25 mM, 100 mM Arginine-HCl, and a pH of 6.00, 6.25, 6.50, 6.75, and 7.00 (see Table 2). Precipitation was measured at 2-8° C. at 5 days, 1 month, and 4 months, or at 40° C. at 1 month and 4 months of storage. Results suggested that increasing Arginine-HCl from a concentration of 0 mM to 25mM universally decreases precipitation out to one month of storage. Further increasing Arginine-HCl concentration to 100 mM reduced precipitation after four months of storage at 40° C. These results suggest that aggregation can be minimized for at least 4 months of storage at 2 to 40° C. if arginine-HCl is at a concentration of about 100 mM in the composition. These results also suggest that aggregation can be minimized for at least 4 months of storage between 2-8° C. if arginine-HCl is at a concentration between 25 and 100 mM in the composition.

Next, experiments were performed to determine the effects of varying CD38-binding fusion protein concentration (5-10 mg/ml), histidine buffer concentration (12.5-107.5 mM), % sucrose (4-8% w/v), Arg-HCl (50-125 mM), polysorbate (PS) type (PS20 vs. PS80), CD38-binding fusion protein to PS ratio (0.5-2.0), and pH (5.5-6.5) (see Table 3 for compositions tested). Compositions were stored at 30° C. Results are summarized in Table 4. Results showed that CD38-binding fusion protein (5-10 mg/ml), % sucrose (4-8% w/v), Arg-HCl (50-125 mM), and polysorbate (PS) type (PS20 vs. PS80) have no impact on composition turbidity, CD38-binding fusion protein basic peak concentration, CD38-binding fusion protein acidic peak concentration, CD38-binding fusion protein higher order aggregation, and CD38-binding fusion protein dimerization (Table 4). No impact, low impact and high impact are defined in the Table 4 legend.

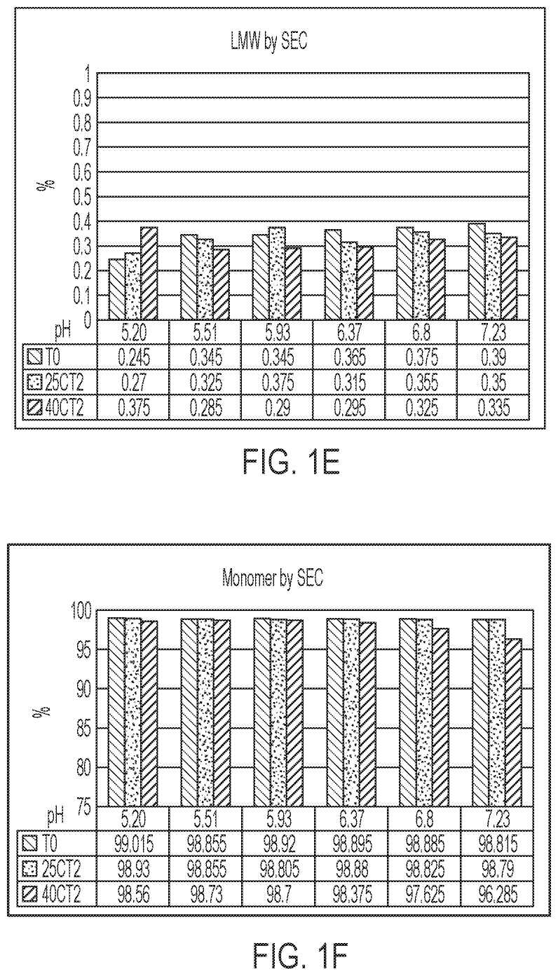

Increasing histidine concentration slightly decreased turbidity, slightly decreased the concentration of the CD38-binding fusion protein basic peak, and slightly increased the concentration of CD38-binding fusion protein acidic peak (FIG. 3). These results suggest that higher histidine concentration promotes better composition turbidity than lower histidine concentration; however, the maximum amount of histidine based on the maximum dose of the CD38-binding fusion protein should not exceed about 50 mM. Increasing CD38-binding fusion protein to PS molar ratio slightly increased turbidity, slightly increased the concentration of the CD38-binding fusion protein basic peak, and slightly decreased the concentration of the CD38-binding fusion protein acidic peak (FIG. 3). These results suggest that a lower CD38-binding fusion protein to PS ratio improves turbidity. Increasing pH decreased turbidity, decreased the concentration of the CD38-binding fusion protein basic peak, and increased the concentration of the CD38-binding fusion protein acidic peak (FIG. 3). These results suggest that having a high composition pH (pH 6.5) is more preferable than having a low composition pH (pH 5.5) because of improvement to turbidity and a decrease in concentration of the basic CD38-binding fusion protein.

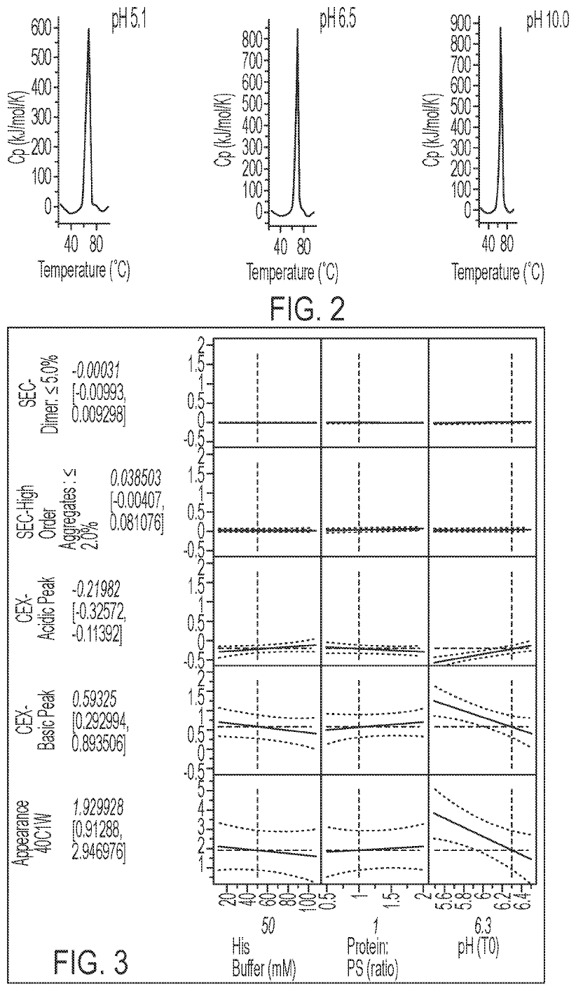

Additional experiments were performed to determine if varying CD38-binding fusion protein (5-15 mg/ml), histidine buffer concentration (40-60 mM), CD38-binding fusion protein to PS80 molar ratio (0.5-1.5), and pH (5.8-6.8) (see Table 5 for compositions tested) affected compositions quality attributes: subvisible particle size and concentration (MFI), turbidity, concentration of the CD38-binding fusion protein main peak, concentration of the CD38-binding fusion protein basic peak, concentration of the CD38-binding fusion protein acidic peak and aggregation. Compositions were stored at 30° C. for and test at 2 weeks and 4 weeks of storage. Results are summarized in Table 6. Results show that CD38-binding fusion protein concentration, histidine buffer concentration, pH, and polysorbate-80/CD38-binding fusion protein ratio of the composition have low or no impact on particle size and concentration or aggregation within the ranges tested (FIG. 4). Increasing CD38-binding fusion protein concentration (5-25 mg/ml) has little to no effect on the main CD38-binding fusion protein, the basic CD38-binding fusion protein, the acidic CD38-binding fusion protein, and CD38-binding fusion protein aggregation (FIG. 4). Overall, this suggests that a lower CD38-binding fusion protein concentration (less than 11 mg/ml) is suitable for the composition.

Increasing histidine buffer concentration (40-60 mM) of the composition has little to no effect on the CD38-binding fusion protein main peak, the CD38-binding fusion protein basic peak, the CD38-binding fusion protein acidic peak, and CD38-binding fusion protein aggregation (FIG. 4). Overall, these results suggest that a histidine buffer concentration between 40 and 60 mM is suitable for the composition.

Increasing pH (6.2-7.1) of the composition slightly increased then slightly decreased the concentration of CD38-binding fusion protein main peak, decreased the concentration of the CD38-binding fusion protein basic peak, increased the concentration of the CD38-binding fusion protein acidic peak, and has a negligible effect on CD38-binding fusion protein aggregation (FIG. 4). Overall, these results suggest that a pH range between 6.3 and 6.9 is suitable for the composition.

Increasing the polysorbate-80/CD38-binding fusion protein molar ratio (0.5 to 1.5) of the composition has little to no effect on the concentration of CD38-binding fusion protein main peak, the concentration of the CD38-binding fusion protein basic peak, the concentration of the CD38-binding fusion protein acidic peak, and on CD38-binding fusion protein aggregation (FIG. 4). Overall, these results suggest that a PS80 to CD38-binding fusion protein molar ratio between 0.5 and 1.5 is suitable for the composition.

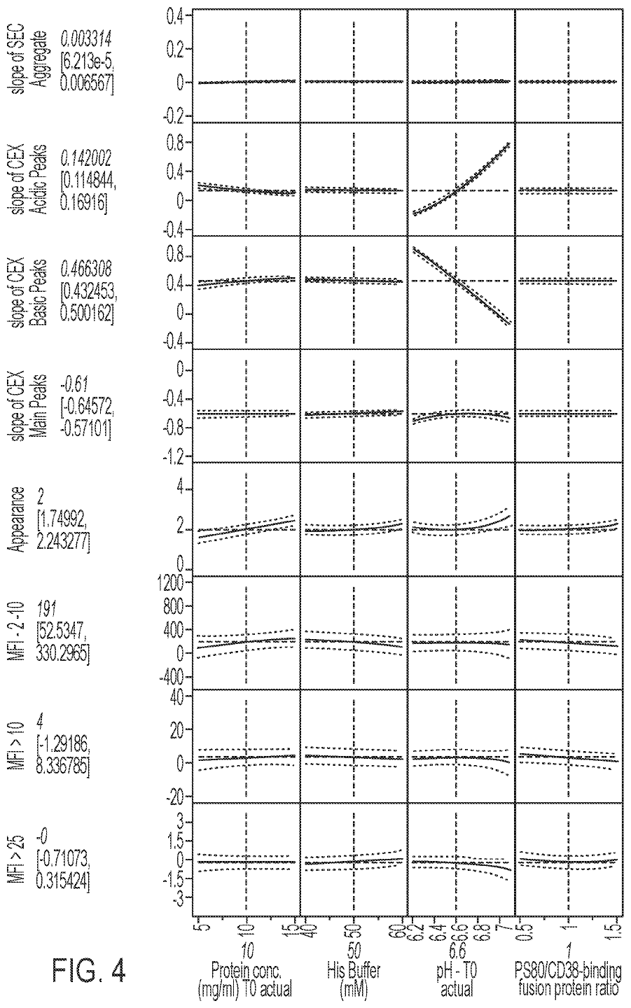

Experiments were further performed to measure pH stability of the composition when varying the concentration of histidine, histidine/HCl, or arginine-HCl by +/−2%. The base composition comprised 10 mg/ml CD38-binding fusion protein, 40 mM histidine, 10 mM histidine HCl, 5% (w/v) sucrose, 100 mM Arginine-HCl, 0.007% (w/v) PS80 and a pH of 6.6. Results showed that pH changes by less than 0.02 after altering the concentration of histidine, histidine/HCl, or arginine-HCl by +/−2% (FIG. 5).

Experiments were further performed to determine how the percentage of PS80 in the composition effected CD38-binding fusion protein visible and subvisible particle formation under mechanical stress conditions. The CD38-binding fusion protein composition comprised 10 mg/ml of the CD38-binding fusion protein containing sequences as provided in Table 1, 50 mM histidine, 5.0% (w/v) sucrose, 100 mM Arginine-HCl, pH 6.6, and 0.005%, 0.007%, 0.01%, or 0.02% PS80 (Table 7). Stress was induced by shaking the sample at room temperature over a period of 120 hours. Visual inspection for formation of visible particles was performed at 0, 2, 4, 6, 8, 24, 48, and 120 hours. Results show that having no PS80 causes extensive foaming of the composition. Results also showed that although the 0.007% PS80 composition had extremely small particles after 24 hours of shaking, the visible particle turbidity was better in comparison to the composition with 0.005% PS80 at same time point. Additionally, compositions with 0.01% PS80 and 0.02% PS80 had no change in visible particle formation after initial inspection at 0 hours.

Effects of PS80 on subvisible particle formation were also quantified using MFI. In these experiments the CD38-binding fusion protein compositions were 10 mg/ml of the CD38-binding fusion protein containing sequences as provided in Table 1, 50 mM histidine, 5.0% w/v sucrose, 100 mM Arginine-HCl, pH 6.6, and 0.005%, 0.007%, 0.01%, or 0.02% PS80 (Table 8). Compositions were shaken for 5 days prior to MFI analysis. MFI was used to count particles between 2-10 μm, ≥10 μm, and ≥25 μm in length. Results show that increasing PS80 concentration generally decreases the number of particles of all sizes with the exception that the composition with 0.01% PS80 has fewer 2-10 μm particles than the composition with 0.02% PS80.

TABLE 2
Arginine-HCl stabilizer testing

| | | Relative area of monomer peak | | | | |
|---|---|---|---|---|---|---|
| | | 2-8° C. | | | 40° C. | |
| | | 5 | 1 | 4 | 1 | 4 |
| Formulation | pH | days | month | months | month | months |
| 100 mM | 6.00 | 97.3 | 97.1 | 97.3 | 92.3 | 93.7 |
| Arg-HCl | 6.25 | 97.2 | 97.2 | 97.5 | 91.2 | N.D. |
| | 6.50 | 97.5 | 97.2 | 97.5 | 87.1 | 89.9 |
| | 6.75 | 97.2 | 97.2 | 97.4 | 83.2 | N.D |
| | 7.00 | 97.2 | 97.1 | 97.4 | 79.1 | 83.8 |
| 25 mM | 6.00 | 97.4 | 97.2 | N.D | 97.4 | High |
| Arg-HCl | 6.25 | 97.4 | 97.2 | | 97.3 | Precipitation = |
| | 6.50 | 97.2 | 97.1 | | 97.0 | N.D |
| | 6.75 | 96.8 | 97.1 | | 96.2 | |
| | 7.00 | 97.2 | 97.1 | | 92.6 | |
| 0 mM | 6.00 | 98.8 | High precipitation = N.D | | | |
| Arg-HCl | 6.25 | High precipitation = N.D | | | | |
| | 6.50 | | | | | |
| | 6.75 | | | | | |
| | 7.00 | | | | | |

TABLE 3
Test 1 - CD38 binding fusion protein formulations

| | CD38 | | | | PS/CD38 | | | |
|---|---|---|---|---|---|---|---|---|
| | binding | His/ | Arg- | Su- | fusion | PS | | |
| Formu- | fusion | HisHCl | HCl | crose | Molar | (mg/ | PS | |
| lation | (mg/ml) | (mM) | (mM) | (%) | Ratio | ml) | Type | pH |
| 1 | 5 | 12.5 | 125 | 8 | 0.5 | 0.02 | PS20 | 6.5 |
| 2 | 5 | 107.5 | 50 | 8 | 0.5 | 0.02 | PS80 | 5.5 |
| 3 | 5 | 107.5 | 125 | 4 | 2 | 0.09 | PS20 | 5.5 |
| 4 | 10 | 12.5 | 125 | 4 | 0.5 | 0.05 | PS80 | 5.5 |
| 5 | 10 | 107.5 | 50 | 4 | 0.5 | 0.04 | PS20 | 6.5 |
| 6 | 5 | 12.5 | 50 | 4 | 2 | 0.09 | PS80 | 6.5 |
| 7 | 10 | 12.5 | 50 | 8 | 2 | 0.17 | PS20 | 5.5 |
| 8 | 10 | 107.5 | 125 | 8 | 2 | 0.18 | PS80 | 6.5 |
| 9 | 10 | 15 | 100 | 5 | 2 | 0.20 | PS80 | 6 |
| 10 | 25 | 15 | 100 | 0 | 0 | 0 | N/A | 6 |

N/A—not applicable

TABLE 4
Summary of results from Table 3 compositions tested.

| | | | Impact on |
|---|---|---|---|
| | Test range | | composition |
| CD38-binding fusion | 5-10 | mg/ml | No impact |
| Histidine buffer | 12.5-107.5 | mM | Low impact |
| Sucrose | 4-8% | (w/v) | No impact |
| Arg-HCl | 50-125 | mM | No impact |

| | | |
|---|---|---|
| PS | PS20 or PS80 | No impact |
| Molar Ratio of Protein: PS | 0.5-2.0 | Low impact |
| pH | 5.5-6.5 | High impact |

*No impact: no change in slope of quality attributes during 4 weeks of storage at 30° C.
*Low impact: minor change in slope of quality attributes during 4 weeks of storage at 30° C.
*High impact: substantial change in slope of quality attributes during 4 weeks of storage at 30° C.

TABLE 5
Experiment 2 - CD38 binding fusion protein formulations

| | CD38- | His/ | | | PS80/CD38- | |
|---|---|---|---|---|---|---|
| | binding | Histidine | Arg- | | binding | |
| | fusion | HCl buffer | HCl | Sucrose | fusion | |
| Formulation | (mg/mL) | (mM) | (mM) | (% w/v) | molar ratio | pH |
| 1 | 5 | 40 | 100 | 5 | 1.5 | 6.2 |
| 2 | 15 | 60 | 100 | 5 | 0.5 | 6.8 |
| 3 | 10 | 50 | 100 | 5 | 1 | 6.5 |
| 4 | 5 | 40 | 100 | 5 | 0.5 | 6.8 |
| 5 | 5 | 60 | 100 | 5 | 0.5 | 6.2 |
| 6 | 15 | 60 | 100 | 5 | 1.5 | 6.2 |
| 7 | 10 | 50 | 100 | 5 | 1 | 6.5 |
| 8 | 15 | 40 | 100 | 5 | 0.5 | 6.2 |
| 9 | 5 | 60 | 100 | 5 | 1.5 | 6.8 |
| 10 | 15 | 40 | 100 | 5 | 1.5 | 6.8 |
| 11 | 15 | 40 | 100 | 5 | 0.5 | 6.8 |
| 12 | 10 | 50 | 100 | 5 | 1 | 6.5 |
| 13 | 5 | 40 | 100 | 5 | 1.5 | 6.8 |
| 14 | 10 | 50 | 100 | 5 | 1 | 6.5 |
| 15 | 5 | 60 | 100 | 5 | 1.5 | 6.2 |
| 16 | 15 | 40 | 100 | 5 | 1.5 | 6.2 |
| 17 | 15 | 60 | 100 | 5 | 1.5 | 6.8 |
| 18 | 5 | 40 | 100 | 5 | 0.5 | 6.2 |
| 19 | 5 | 60 | 100 | 5 | 0.5 | 6.8 |
| 20 | 15 | 60 | 100 | 5 | 0.5 | 6.2 |
| 21 | 10 | 30 | 100 | 5 | 1 | 6.5 |
| 22 | 20 | 50 | 100 | 5 | 1 | 6.5 |
| 23 | 10 | 50 | 100 | 5 | 0 | 6.5 |
| 24 | 10 | 50 | 100 | 5 | 1 | 6.5 |
| 25 | 10 | 50 | 100 | 5 | 1 | 5.9 |
| 26 | 10 | 50 | 100 | 5 | 2 | 6.5 |
| 27 | 10 | 70 | 100 | 5 | 1 | 6.5 |
| 28 | 0 | 50 | 100 | 5 | 1 | 6.5 |
| 29 | 10 | 50 | 100 | 5 | 1 | 7.1 |
| 30 | 10 | 50 | 100 | 5 | 1 | 6.5 |

TABLE 6
Summary of results from Table 5 compositions tested.

| | |
|---|---|
| | Impact on |
| Test range | composition |

| | | | |
|---|---|---|---|
| CD38-binding fusion | 5-15 | mg/ml | Low impact |
| Histidine buffer | 40-60 | mM | No impact |

| | | |
|---|---|---|
| Ratio of Protein to PS80 | 0.5-1.5 | Low impact |
| pH | 5.8-7.1 | High impact |

*No impact: no change in slope of quality attributes during 4 weeks of storage at 30° C.
*Low impact: minor change in slope of quality attributes during 4 weeks of storage at 30° C.
*High impact: substantial change in slope of quality attributes during 4 weeks of storage at 30° C.

TABLE 7
Results of Shaking Study Initiated with CD38-binding fusion protein composition

| Drug Product | | | | | | | | |
|---|---|---|---|---|---|---|---|---|
| (DP) samples | | | | | | | | |
| shaken at | | | | | | | | |
| controlled | | | | | | | | |
| room | | | | | | | | |
| temperature * | Initial | 2 hrs | 4 hrs | 6 hrs | 8 hrs | 24 hrs | 48 hrs | 120 hrs |
| No PS80 | — | Extensive | N/A | N/A | N/A | N/A | N/A | N/A |
| | | foaming. | | | | | | |
| | | Sample | | | | | | |
| | | not | | | | | | |
| | | agitated | | | | | | |
| | | further. | | | | | | |
| With 0.005% | — | No | No | One | One | + | No | + |
| PS80 | | change | change | small | small | (extremely | change | (extremely |
| | | | | fiber | fiber | small) and | from | small) and |
| | | | | | | visible only | 24 hrs | visible |
| | | | | | | under | | only under |
| | | | | | | Tyndall | | Tyndall |
| | | | | | | light | | light |
| With 0.007% | One | No | No | No | No | + | No | No |
| PS80** | fiber | change | change | change | change | (extremely | change | change |
| | | | | | | small)¹ and | from | from |
| | | | | | | visible only | 24 hrs | 48 hrs |
| | | | | | | under | | |
| | | | | | | Tyndall | | |
| | | | | | | light | | |
| With 0.01% | Two | No | No | No | No | No | No | No |
| PS80 | small | change | change | change | change | change | change | change |
| | fibers | | | | | | | |
| | (red and | | | | | | | |
| | white) | | | | | | | |
| | and one | | | | | | | |
| | small | | | | | | | |
| | translucent | | | | | | | |
| | spec | | | | | | | |
| With 0.02% | One | No | No | No | No | No | No | No |
| PS80 | small | change | change | change | change | change | change | change |
| | fiber | | | | | | | |
| | and one | | | | | | | |
| | small | | | | | | | |
| | translucent | | | | | | | |
| | spec | | | | | | | |

* The CD38-binding fusion protein composition comprised 10 mg/ml CD38-binding fusion protein, 50 mM histidine, 5% w/v sucrose, 100 mM Arg-HCl, 0.02% w/v PS80, and pH 6.6.
**Although DP with 0.007% had extremely small particles after 24 hrs of shaking, the visible particle turbidity was better in comparison to DP with 0.005% PS80 at same time point. The extent of foaming was inversely proportional to decrease in PS80 concentration up to 0.01% PS80 (i.e., less PS80 resulted in more foaming).
— No particles
+ Very few fibers and/or particles
N/A Experiment stopped for sample without PS80

TABLE 8
The Number of Subvisible Particles/mL in CD38-binding fusion protein
compositions after 5 days of shaking at room temperature.

| Sample | Total | Average | 2-10 μm | Average | ≥10 μm | Average | ≥25 μm | Average |
|---|---|---|---|---|---|---|---|---|
| Water | 54 | 57 | 46 | 49 | 8 | 8 | 0 | 2 |
| | 59 | | 51 | | 8 | | 3 | |
| 0.005% | 3259 | 3159 | 3103 | 3063 | 156 | 95 | 18 | 7 |
| PS80 | 3313 | | 3228 | | 84 | | 3 | |
| (shaken for | 2904 | | 2858 | | 46 | | 0 | |
| 5 days) | | | | | | | | |
| 0.007% | 2958 | 2384 | 2836 | 2310 | 122 | 75 | 20 | 9 |
| PS80 | 2127 | | 2096 | | 31 | | 3 | |
| (shaken for | 2068 | | 1997 | | 71 | | 5 | |
| 5 days) | | | | | | | | |
| 0.01% | 1015 | 1062 | 949 | 1020 | 66 | 42 | 13 | 6 |
| PS80 | 1063 | | 1030 | | 33 | | 5 | |
| (shaken for | 1107 | | 1081 | | 26 | | 0 | |
| 5 days) | | | | | | | | |
| 0.02% | 1675 | 1656 | 1622 | 1617 | 54 | 39 | 15 | 7 |
| PS80 | 1970 | | 1942 | | 28 | | 3 | |
| (shaken for | 1324 | | 1288 | | 36 | | 3 | |
| 5 days) | | | | | | | | |

### Example 2—Stability of CD38-Binding Fusion Protein Compositions

The (CD38-binding fusion protein) lyophilized composition may be stored long-term at 5±3° C. and formulated as 10 mg/ml protein in 50 mM histidine/histidine hydrochloride, 100 mM arginine hydrochloride with 5% (w/v) sucrose and 0.02% (w/v) polysorbate 80 at pH 6.6.

The quality attributes of this formulation were evaluated on stability at long term storage (5±3° C.), accelerated (25±2° C.) and stress (40±2° C.) storage conditions. Test methods employed in the study were chosen to assess the purity, potency, and other quality attributes of the drug product. Testing included appearance (lyophilized cake and reconstituted liquid), turbidity and color (reconstituted liquid), reconstitution time, size exclusion chromatography (SE-UPLC), imaged capillary isoelectric focusing (icIEF), potency, protein concentration, pH, moisture content, and additional characterization methods of sub-visible particle (SVP) analysis by microflow imaging (MFI) and PS80 content determination.

The stability data at long term (5° C.±3° C.) for lot 1, lot 2 and lot 3 meet the acceptance criteria with no significant change or trends to date in general properties, content, purity and impurities, and microbiological quality up to the 12-month, 6-month and 1-month time points, respectively. A degradation rate analyses model was used to determine the shelf-life of the CD38-binding fusion protein composition. The degradation rate model (with 95% confidence interval) shows that SEC (% Main Peak), reduced CE-SDS (% H+L), non-reduced CE-SDS (% IgG), and icIEF (% Major Isoform, % Acidic Peak, % Basic Peak areas) will remain within the acceptance criteria of the specification SPEC-0014566 through 24 months at the long-term storage condition of 5° C.±3° C. (data not shown).

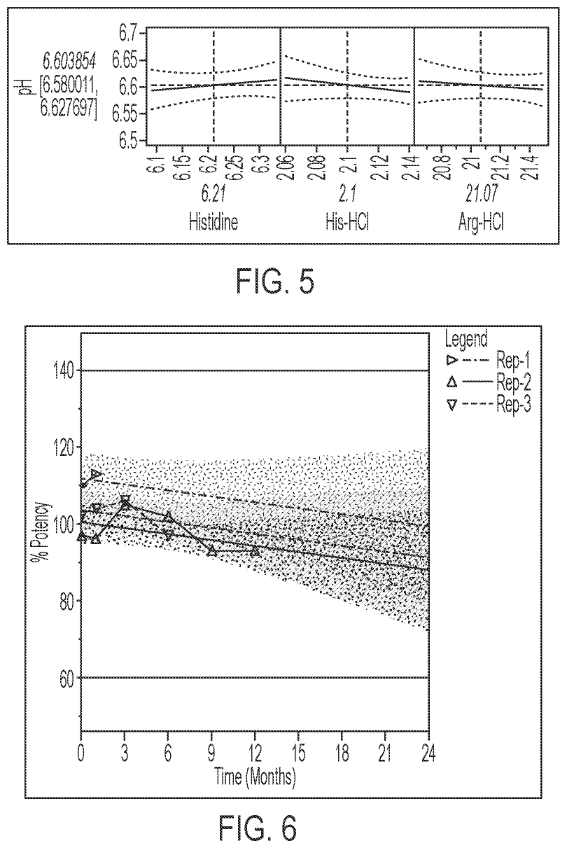

Assays were also performed to measure potency. Results from a cell based potency assays show that, after storage at 5° C.±3° C., CD38-binding fusion protein composition protein decreases by no more than 10% over 12 months of storage (FIG. 6). The potency was analyzed using the degradation rate analyses model in JMP to determine the expected shelf life of the CD38-fusion protein composition. Based on the degradation rate model (with 95% confidence interval), this CD38-fusion protein composition will remain within the acceptance criteria for potency through 24 months at the long-term storage condition of 5° C.±3° C.

The stability data at accelerated (25° C.±2° C./60% RH (relative humidity)±5% RH) condition for lot 1, lot 2 and lot 3 meet the acceptance criteria with no significant change or trends to date for all the quality attributes tested up to the 6-month and 1-month time points, respectively. However, at the 3 months timepoint of lot 2, a relative potency value of 146% was reported, which does not meet the long term acceptance criteria limit of 60%-140%. However, the subsequent time point of 6 months, the relative potency result was within the long-term specification, and therefore, the potency result at the 3-month time point is considered a one-time event and future time points potency data will be closely monitored. Moreover, all other physicochemical quality attributes showed no trends at the 6-month timepoint at the accelerated condition.

The stability data at stress (40±2° C./75±5% RH (relative humidity)) condition for lot 1, lot 2 and lot 3 meet the long term acceptance criteria to date for all the quality attributes tested up to the 3-month and 1-month time points, respectively. Based on the development stability data at the stress condition, there is a slight decreasing trend for the icIEF % Main peak area values with a corresponding increase in the % Basic species peak area at the stress condition. The relative potency result for lot 2 at the 3-month time point was 154%, which is outside the long term acceptable criteria similar to the result obtained at the 3-month, accelerated condition for this lot discussed above.

Overall, these experiments demonstrate that the CD38-binding fusion protein composition is stable for at least 12 months of storage at 5° C.±3° C. and is modeled to be stable for 24 months.

What is claimed is:

1. A composition comprising a CD38-binding fusion protein, a buffer, a tonicity agent, a stabilizer, and a surfactant, wherein the CD38-binding fusion protein comprises an anti-CD38 antibody fused to an attenuated interferon alpha-2b.

2. The composition of claim 1, wherein the anti-CD38 antibody comprises a heavy chain complementarity determining region 1 (CDR-H1) comprising the amino acid sequence of SEQ ID NO: 1, a heavy chain complementarity determining region 2 (CDR-H2) comprising the amino acid sequence of SEQ ID NO: 2, a heavy chain complementarity determining region 3 (CDR-H3) comprising the amino acid sequence of SEQ ID NO: 3, a light chain complementarity determining region 1 (CDR-L1) comprising the amino acid sequence of SEQ ID NO: 4, a light chain complementarity determining region 2 (CDR-L2) comprising the amino acid sequence of SEQ ID NO: 5, a light chain complementarity determining region 3 (CDR-L3) comprising the amino acid sequence of SEQ ID NO: 6.

3. The composition of claim 1 or claim 2, wherein the anti-CD38 antibody comprises a heavy chain variable region comprising the amino acid sequence of SEQ ID NO: 7 and a light chain variable region comprising the amino acid sequence of SEQ ID NO: 8.

4. The composition of any one of claims 1-3, wherein the anti-CD38 antibody comprises a human IgG4 constant region.

5. The composition of claim 4, wherein the human IgG4 constant region comprises a proline at position 228 according to the EU numbering system.

6. The composition of claim 5, wherein the human IgG4 constant region further comprises a tyrosine at position 252, a threonine at position 254, and a glutamic acid at position 256 of the constant region according to the EU numbering system.

7. The composition of any one of claims 1-6, wherein the anti-CD38 antibody comprises a heavy chain comprising the amino acid sequence of SEQ ID NO: 9 and a light chain comprising the amino acid sequence of SEQ ID NO: 10.

8. The composition of any one of claims 1-7, wherein the attenuated interferon alpha-2b comprises T106A and A145D mutations relative to an interferon alpha-2b comprising the amino acid sequence of SEQ ID NO: 11.

9. The composition of any one of claims 1-8, wherein the attenuated interferon alpha-2b comprises the amino acid sequence of SEQ ID NO: 12.

10. The composition of any one of claims 7-9, wherein the attenuated interferon alpha-2b is fused to the C-terminus of the heavy chain.

11. The composition of claim 10, wherein the CD38-binding fusion protein comprises a heavy chain comprising the amino acid sequence of SEQ ID NO: 13 and a light chain comprising the amino acid sequence of SEQ ID NO: 10.

12. The composition of any one of claims 1-11, wherein the composition comprises the CD38-binding fusion protein at a concentration of about 8.5-100 mg/ml.

13. The composition of any one of claims 1-11, wherein the composition comprises the CD38-binding fusion protein at a concentration of about 30-100 mg/ml.

14. The composition of any one of claims 1-11, wherein the composition comprises the CD38-binding fusion protein at a concentration of about 30-70 mg/ml.

15. The composition of any one of claims 1-11, wherein the composition comprises the CD38-binding fusion protein at a concentration of about 30 mg/ml.

16. The composition of any one of claims 1-11, wherein the composition comprises the CD38-binding fusion protein at a concentration of about 40 mg/ml.

17. The composition of any one of claims 1-11, wherein the composition comprises the CD38-binding fusion protein at a concentration of about 60 mg/ml.

18. The composition of any one of claims 1-11, wherein the composition comprises the CD38-binding fusion protein at a concentration of about 80 mg/ml.

19. The composition of any one of claims 1-11, wherein the composition comprises the CD38-binding fusion protein at a concentration of about 8.5-11.5 mg/ml.

20. The composition of any one of claims 1-11, wherein the composition comprises the CD38-binding fusion protein at a concentration of about 10 mg/ml.

21. The composition of any one of claims 1-20, wherein the buffer comprises histidine and histidine-HCl.

22. The composition of any one of claims 1-21, wherein the composition comprises a total histidine at a concentration of about 50-75 mM.

23. The composition of any one of claims 1-21, wherein the composition comprises a total histidine at a concentration of about 50 mM.

24. The composition of any one of claims 1-23, wherein the tonicity agent is arginine-HCl.

25. The composition of any one of claims 1-24, wherein the composition comprises arginine-HCl at a concentration of about 100-150 mM.

26. The composition of any one of claims 1-24, wherein the composition comprises arginine-HCl at a concentration of about 100 mM.

27. The composition of any one of claims 1-25, wherein the stabilizer is a carbohydrate.

28. The composition of any one of claims 1-25, wherein the stabilizer is a hexose.

29. The composition of any one of claims 1-25, wherein the stabilizer is a trehalose.

30. The composition of any one of claims 1-25, wherein the stabilizer is sucrose.

31. The composition of any one of claims 1-30, wherein the composition comprises stabilizer at a concentration of about 50-100 mg/ml.

32. The composition of any one of claims 1-30, wherein the composition comprises stabilizer at a concentration of about 50 mg/ml.

33. The composition of any one of claims 1-30, wherein the composition comprises sucrose at a concentration of about 50 mg/ml.

34. The composition of any one of claims 1-33, wherein the surfactant is polysorbate 80 (PS80).

35. The composition of any one of claims 1-34, wherein the composition comprises PS80 at a concentration of about 0.1-0.6 mg/ml.

36. The composition of any one of claims 1-34, wherein the composition comprises PS80 at a concentration of about 0.2 mg/ml.

37. The composition of any one of claims 1-36, wherein the composition has a pH of 6.0-7.0.

38. The composition of any one of claims 1-36, wherein the composition has a pH of 6.5-6.7.

39. The composition of any one of claims 1-37, wherein the composition has a pH of 6.6.

40. A composition comprising 10 mg/ml of a CD38-binding fusion protein, 50 mM of histidine, 100 mM of arginine, 50 mg/ml of sucrose, and 0.2 mg/ml of polysorbate 80 (PS80), wherein the composition has a pH of 6.6, wherein the CD38-binding fusion protein comprises an anti-CD38 antibody comprising a heavy chain and a light chain, wherein the heavy chain comprises the amino acid sequence of SEQ ID NO: 9 and is fused to an attenuated interferon alpha-2b comprising the amino acid sequence of SEQ ID NO: 12, and wherein the light chain comprises the amino acid sequence of SEQ ID NO: 10.

41. A composition comprising 30-100 mg/ml of a CD38-binding fusion protein, 50-75 mM of histidine, 100-150 mM of arginine, 50-100 mg/ml of sucrose, 0.1 to 0.6 mg/ml of polysorbate 80 (PS80), wherein the composition has a pH of 6.0-7.0, wherein the CD38-binding fusion protein comprises an anti-CD38 antibody comprising a heavy chain and a light chain, wherein the heavy chain comprises the amino acid sequence of SEQ ID NO: 9 and is fused to an attenuated interferon alpha-2b comprising the amino acid sequence of SEQ ID NO: 12, and wherein the light chain comprises the amino acid sequence of SEQ ID NO: 10.

42. The composition of claim 41, comprising 40 mg/ml of the CD38 binding fusion protein.

43. The composition of claim 41, comprising 60 mg/ml of the CD38 binding fusion protein.

44. The composition of claim 41, comprising 80 mg/ml of the CD38 binding fusion protein.

45. The composition of any one of claims claim 40-44, wherein the CD38-binding fusion protein comprises a heavy chain comprising the amino acid sequence of SEQ ID NO: 13 and a light chain comprising the amino acid sequence of SEQ ID NO: 10.

46. The composition of any one of claims 1-45, wherein the composition is lyophilized.

47. The composition of any one of claims 1-46, wherein the composition is in dosage unit form.

48. A method of treating a CD38-expressing cancer, the method comprising administering to a subject in need thereof an effective amount of the composition of any one of claims 1-47.

49. The method of claim 48, wherein the CD38-expressing cancer is B-cell lymphoma, multiple myeloma, Waldenstrom's macroglobulinemia, non-Hodgkin's lymphoma, chronic myelogenous leukemia, chronic lymphocytic leukemia, or acute lymphocytic leukemia.

50. The method of claim 48 or claim 49, wherein the CD38-expressing cancer is multiple myeloma.

51. The method of claim 50, wherein the multiple myeloma is refractory multiple myeloma.

52. The method of any one of claims 48-51, wherein the subject is human.

53. The method of any one of claims 48-52, further comprising administering to the subject lenalidomide or pomalidomide.

54. The composition of any one of claims 1-53 for use in a method for treating a CD38-expressing cancer in a subject.

55. The composition of claim 54, wherein the subject is receiving treatment with lenalidomide or pomalidomide.

56. The method of any one of claims 48-53, further comprising administering to the subject a CD47 antagonist.

57. The composition of claim 54, wherein the subject is receiving treatment with a CD47 antagonist.


SEQUENCE LISTING (15 sequences)

SEQ ID NO: 1 - protein, 5 aa, Synthetic construct
DSVMN

SEQ ID NO: 2 - protein, 17 aa, Synthetic construct
WIDPEYGRTDVAEKFQG

SEQ ID NO: 3 - protein, 11 aa, Synthetic construct
TKYNSGYGFPY

SEQ ID NO: 4 - protein, 11 aa, Synthetic construct
KASQNVDSDVD

SEQ ID NO: 5 - protein, 7 aa, Synthetic construct
KASNDYT

SEQ ID NO: 6 - protein, 9 aa, Synthetic construct
MQSNTHPRT

SEQ ID NO: 7 - protein, 120 aa, Synthetic construct
EVQLVQSGAEVKKPGATVKISCKVSGYTFTDSVMNWVQQAPGKGLEWMGWIDPEYGRTDVAEKFQGRVTITADTSTDTAYMELSSLRSEDTAVYYCARTKYNSGYGFPYWGQGTTVTVSS

SEQ ID NO: 8 - protein, 108 aa, Synthetic construct
DIQMTQSPSSLSASVGDRVTITCKASQNVDSDVDWYQQKPGKAPKLLIYKASNDYTGVPSRFSGSGSGTDFTFTISSLQPEDIATYYCMQSNTHPRTFGGGTKVEIKR

SEQ ID NO: 9 - protein, 447 aa, Synthetic construct
EVQLVQSGAEVKKPGATVKISCKVSGYTFTDSVMNWVQQAPGKGLEWMGWIDPEYGRTDVAEKFQGRVTITADTSTDTAYMELSSLRSEDTAVYYCARTKYNSGYGFPYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 214 aa, Synthetic construct
DIQMTQSPSSLSASVGDRVTITCKASQNVDSDVDWYQQKPGKAPKLLIYKASNDYTGVPSRFSGSGSGTDFTFTISSLQPEDIATYYCMQSNTHPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 165 aa, Homo sapiens [sequence omitted]

SEQ ID NO: 12 - protein, 165 aa, Synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVAETPLMKEDSILAVRKYFQRITLYLKEKKYSPCAWEVVRDEIMRSFSLSTNLQESLRSKE

SEQ ID NO: 13 - protein, 612 aa, Synthetic construct
EVQLVQSGAEVKKPGATVKISCKVSGYTFTDSVMNWVQQAPGKGLEWMGWIDPEYGRTDVAEKFQGRVTITADTSTDTAYMELSSLRSEDTAVYYCARTKYNSGYGFPYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVAETPLMKEDSILAVRKYFQRITLYLKEKKYSPCAWEVVRDEIMRSFSLSTNLQESLRSKE

SEQ ID NO: 14 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 15 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK